(19) United States
(12) Patent Application Publication
(10) Pub. No.: US 2013/0129648 A1
(43) Pub. Date: May 23, 2013

(54) SILICONE BASED COSMETIC COMPOSITIONS AND USES THEREOF

(71) Applicants: Nghi Van Nguyen, Edison, NJ (US); Sawa Hashimoto, Garwood, NJ (US); Siliu Tan, Westfield, NJ (US)

(72) Inventors: Nghi Van Nguyen, Edison, NJ (US); Sawa Hashimoto, Garwood, NJ (US); Siliu Tan, Westfield, NJ (US)

(73) Assignee: L'OREAL, Paris (FR)

(21) Appl. No.: 13/814,963

(22) PCT Filed: Aug. 10, 2011
(86) PCT No.: PCT/US11/47179

§ 371 Date: Feb. 8, 2013

Related U.S. Application Data
(60) Provisional application No. 61/372,220, filed on Aug. 10, 2010.
(60) Provisional application No. 61/372,227, filed on Aug. 10, 2010.
(60) Provisional application No. 61/372,144, filed on Aug. 10, 2010.
(60) Provisional application No. 61/372,143, filed on Aug. 10, 2010.
(60) Provisional application No. 61/372,139, filed on Aug. 10, 2010.

(51) Int. Cl. A61K 8/898; A61Q 5/12; A61Q 19/04; A61K 8/896; A61Q 19/00; A61Q 19/08; A61Q 17/04; A61Q 5/00; A61Q 19/02

(52) U.S. Cl. CPC A61K 8/898; A61Q 5/00; A61Q 5/12; A61Q 19/04; A61Q 19/02; A61Q 19/007; A61Q 19/08; A61Q 17/04; A61K 8/896
USPC 424/59; 424/70.122; 424/62; 424/70.9; 424/70.6

(57) ABSTRACT

The present disclosure relates to silicone based cosmetic compositions comprising at least at least one silicone amine, at least one anionic silicone and a cosmetically acceptable carrier such as water and other components. The compositions are useful for treating keratinous substrates such as hair, for example, for styling, conditioning and/or improving the manageability of hair. The present disclosure also relates to silicone based hair coloring compositions comprising at least one silicone amine, at least one anionic silicone, at least one colorant, a cosmetically acceptable carrier; and optionally, at least one oxidizing agent. The compositions are useful for coloring hair.

### FIELD OF THE DISCLOSURE

The present disclosure relates to silicone based cosmetic compositions comprising a silicone amine and an anionic silicone, and methods for using the cosmetic compositions. The present disclosure additionally relates to silicone based coloring compositions and uses thereof for artificially coloring and/or preserving artificial color in/on a keratinous substrate.

### BACKGROUND

An essential constituent of the body is keratin, which plays a decisive role in determining the properties of hair, skin, scalp, lips and nails. The keratin is often affected or damaged by external influences and/or adverse environmental conditions. For example, high or low humidity conditions can adversely affect the skin in terms of moisturization, suppleness and smoothness. Other external influences such as solar irradiation (irradiation with UV light), air pollutants, and mechanical or physical rubbing/scrubbing of the skin can also damage or irritate the skin, thereby affecting the condition and appearance of the skin and/or scalp.

Solar irradiation (irradiation with UV light) can also lead to a reduction in the mechanical strength of the hair and to a bleaching of the natural and artificial hair color. Chlorinated water in swimming pools etc. causes oxidation of the keratin, which can lead to a loss of protein. Moreover, human hair is often subjected to chemical hair treatments, such as treatments with permanent waving or hair straightening compositions, hair colorants, bleaches, shampoos, etc. Additionally, hair is subjected to regular washing, combing, brushing and blow drying. Just like UVA irradiation, these treatments can change the original properties of the hair, damage the hair and/or produce color-changing effects on the hair. For example, bleaching generally leads to hair damage, where the melanine pigments in the cuticle cells and the cortex cells of the hair are degraded by complex chemical reactions.

Damaged hair becomes rough, brittle and more difficult to manage, comb and/or disentangle. Loss of shine can also result from exposure to UV light and high humidity and the artificial color on hair fades from exposure to UV light and washing. Additionally, the noxae involved in hair chemical treatments cannot be completely eliminated after the first rinsing so that the result is ultimately insidious oxidation and consequently the specified hair damage and/or undesirable color changes in both dyed and naturally colored hair.

In addition, high or low humidity conditions result in reduced manageability making it more difficult to comb or groom and style or shape the hair. Under high humidity conditions, hair can become frizzy, more porous and more sensitive to the effect of moisture and therefore requires longer drying times. Its resistance and tear strength are reduced and its extensibility is altered. Under low humidity conditions, the hair becomes dry. It is thus desirable to apply a composition on the hair which will help keep moisture out of the hair or help retain moisture in the hair at various humidity conditions.

It has been surprisingly and unexpectedly found that the silicone based cosmetic compositions of the present disclosure provide cosmetic benefits to hair and skin and can be used for beneficially coating, treating, protecting and/or improving the condition and quality of hair and skin.

Imparting a color change or color effect to hair can be done using permanent, semi-permanent, and temporary hair coloring products. Many permanent hair coloring products use oxidative hair color that bleaches the melanin found in the hair shaft as well as imparting color. Activated peroxide in the oxidative dye composition provides a bleaching effect while the oxidative dye molecules penetrate the hair shaft and polymerize therein. While hair that is oxidatively colored provides a certain permanence, re-coloring every four to six weeks may be necessary due to new hair growth. Oxidative hair color is sold in the form of a two component kit. The reactivity of the oxidative dye and the oxidizing agent means that the two ingredients cannot be formulated into a single composition. Rather, the kits have one container filled with an aqueous composition in the liquid, gel, or cream form that contains the oxidative dyes and a second container filled with a developer composition that contains an oxidizing agent, usually hydrogen peroxide. The two containers are combined immediately prior to use and applied to hair. The oxidizing agent and dyes react when mixed. The mixture is applied to hair for an appropriate period of time, generally 20 to 60 minutes, then rinsed off with water. Permanent hair color is very versatile and long lasting in the colors and effects it provides, and it is only with permanent hair color that one can color hair to a shade that is lighter than the natural color.

Semi-permanent hair color generally provides more lasting color than temporary dyes but without the permanence and commitment of oxidative color. Semi-permanent color is a single component product containing direct dyes. Many of such products contain two types of dyes: one of smaller molecular size (such as nitrophenylene diamines and nitroaminophenols) that are capable of penetrating the hair shaft and being retained within. While smaller molecular size permits these dyes to more easily penetrate the hair shaft. The second type of dye used in semi-permanent hair color has a larger molecular size and is too large to penetrate the hair shaft of normal virgin hair. However, such larger dye molecules easily penetrate porous and damaged hair where they are often retained due to their larger size. Accordingly, the balancing of the small and large dye molecules found in many semi-permanent products provides color with a uniform and durable effect. Semi-permanent color provides excellent gray blending and good color retention.

Temporary hair color may employ pigments and natural colorants (e.g., vegetable-derived) is often found in the rinse form, and typically lasts for one shampoo. Such hair color is often used when special effects are desired. Temporary color simply coats the hair shaft with colorants that are too large to penetrate its outer surface. Minor penetration of the hair shaft may occur in individuals with damaged or porous hair, but such color application rarely lasts through more than one or two shampoos.

Thus, it is an object of the present disclosure to provide a silicone based composition for treating keratinous substrates such as the hair, skin and/or the scalp wherein the composition can provide cosmetic benefits to and help protect the keratinous substrates from external influences or improve the condition of the keratinous substrates. It is an additional object of the present disclosure to provide hair coloring compositions and methods for coloring hair including methods for simultaneously washing and depositing color onto the hair.

### SUMMARY OF THE INVENTION

The present disclosure relates to silicone based cosmetic compositions comprising:
(a) at least one silicone amine;
(b) at least one anionic silicone; and
(c) a cosmetically acceptable carrier.

The cosmetic composition may further comprise at least one auxiliary ingredient such as a film forming agent, a surfactant, a conditioning agent, and/or a skin active agent. The cosmetic composition can be used, for example, to beneficially coat keratinous substrates such as the hair and/or skin. Methods of beneficially coating keratinous substrates include providing (1) shine to hair; (2) conditioning/manageability to hair; (3) anti-frizz to hair; (4) styling to hair; (5) color retention/fade resistance to artificially colored hair, (6) moisture retention to skin, scalp or hair; (7) sun protection; and (8) skin care benefits such as, for example, anti-aging, whitening/depigmentation, anti-acne, etc.

The present disclosure also relates to silicone based hair coloring compositions comprising:
(a) at least one silicone amine;
(b) at least one anionic silicone;
(c) at least one colorant;
(d) a cosmetically acceptable carrier; and
(e) optionally, at least one oxidizing agent.

The hair coloring compositions may also optionally include a conditioning agent.

The hair coloring compositions provide enhanced coloring effects to the hair such as greater color deposit, and more vibrant and intense color to hair than typical hair coloring methods. Thus, the hair coloring compositions can be used to (1) increase color deposit on artificially colored hair; (2) boost the intensity of a colorant in artificially colored hair; (3) improve the fade resistance of artificial color on hair; and (4) highlight hair.

The present disclosure also relates to a method for simultaneously washing and depositing color onto hair using the above-mentioned silicon-based hair coloring compositions. The method comprises (a) applying a hair coloring composition described above to hair, and (b) rinsing the composition from the hair.

Due to the conditioning properties of the silicone amine/anionic silicone combination, the compositions of the instant disclosure impart a softness to the hair and prevent the drying and breaking that can occur in nature and with the use of oxidative dyes.

Examples of silicone amines that are useful in the hair coloring compositions include but are not limited to amodimethicone, trimethylsilylamodimethicone, aminopropyl phenyltrimethicone, PEG-7 amodimethicone, aminopropyl triethyoxysilane, alkyl modified amino silicone, amino silanes, PEG-8 amodimethicone, bis-isobutyl PEG-14/amodimethicone copolymer, bis-isobutyl PEG-15/amodimethicone copolymer, bis-isobutyl PEG/PPG-20/35/amodimethicone copolymer, Trideceth-9 PG-Amodimethicone and mixtures thereof.

In one embodiment, the silicone amine is amodimethicone. In another embodiment, the silicone amine chosen from polyethylene glycol derivatives of amodimethicone such as PEG-7 amodimethicone and PEG-8 amodimethicone and modified aminosiloxanes such as Trideceth-9 PG-Amodimethicone (commercially available as SilCare Silicone SEA sold by Clariant International, LTD).

The cosmetic compositions typically comprise a silicone amine in an amount of from about 1% to about 90%, from about 1% to about 75%, from about 1% to about 50%, from about 1% to about 25% or from about 1% to about 15% by weight based on the total weight of the cosmetic composition.

Examples of anionic silicones that are useful in the cosmetic compositions include but are not limited to silicone carboxylates, silicone phosphates, silicone sulfates, silicone sulfosuccinates, silicone sulfonates, and mixtures thereof. In one embodiment, the anionic silicone is a silicone carboxylate (such as dimethicone PEG-7 phthalate and/or dimethicone PEG-7 succinate) or a silicone phosphate (such as dimethicone PEG-7 phosphate, dimethicone PEG-8 phosphate, dimethicone PEG-10 phosphate, potassium dimethicone PEG-7 panthenyl phosphate, dimethicone PEG/PPG-7/4 phosphate, dimethicone PEG/PPG-12/4 phosphate, and mixtures thereof). In another embodiment, the (b) anionic silicone is dimethicone PEG-8 phosphate.

The cosmetic compositions typically comprise an anionic silicone in an amount of from about 1% to about 90%, from about 1% to about 75%, from about 1% to about 50%, from about 1% to about 25% or from about 1% to about 15% by weight based on the total weight of the cosmetic composition.

In one embodiment, the ratio of silicone amine to anionic silicone is from about 1:20 to about 20:1, from about 1:10 to about 10:1, from about 5:1 to about 1:5, from about 3:1 to about 1:3, about 2:1, or about 1:1.

In hair coloring compositions, the colorant can be any colorant appropriate for use on hair. For example, the colorant may be selected from oxidative dyes, direct dyes, pigments, natural colorants, and mixtures thereof. In one embodiment, the colorant includes an oxidative dye, and may optionally further include at least one primary dye intermediate and/or a coupler compound. Oxidative dyes are typically present in an amount from about 0.0001% to about 12% by weight based on the total weight of the coloring composition and may include peroxides, persulfates, perborates, percarbonates, alkali metal bromates, redox enzymes, ferricyanides, and mixtures thereof. In one embodiment, the oxidizing agent includes hydrogen peroxide. In another embodiment the oxidizing agent is present in an amount of at least about 1.0% by weight based on the total weight of the coloring composition. In another embodiment, the colorant includes a direct dye. A direct dye is typically present in an amount from about 0.0001% to about 30% by weight based on the total weight of the coloring composition. The colorant may also include a pigment. A pigment is typically present in an amount from about 0.5% to about 40% by weight based on the total weight of the coloring composition.

The hair coloring compositions can further include at least one conditioning agent. Representative conditioning agents include a polyalkyl siloxane, a polyaryl siloxane, a polyalkylaryl siloxane, a polyether siloxane copolymer, or a mixtures thereof. For example, the conditioning agent can be a polyalkyl siloxane, a polyaryl siloxane, a polyalkylaryl siloxane, a polyether siloxane copolymer, and mixtures thereof. In one embodiment, the conditioning agent is a polydimethyl siloxane, a polydiethyl siloxane, a polydipropyl siloxane, a polymethylethyl siloxane, a polymethylpropylsiloxane, a polymethylphenylsiloxane, or a mixture thereof. In another embodiment, the at least on conditioning agent is present in an amount from about 1% to about 90.0% by weight based on the total weight of the coloring composition.

In addition to coloring hair, the hair coloring compositions can be used in a method for (1) increasing color deposit on artificially colored hair; (2) boosting the intensity of a colorant in artificially colored hair; (3) improving the fade resistance of artificial color on hair; and (4) highlighting hair. The present disclosure also relates to kits useful for applying artificial color to keratinous substrates and preserving artificial coloring on the keratinous substrates. The kits comprise a first composition comprising at least one colorant for imparting artificial colorant to a keratinous substrate and a second composition comprising at least one silicone amine, at least one anionic silicone, optionally, at least one surfactant, and a cosmetically acceptable carrier.

Cosmetically acceptable carriers include water and/or other cosmetically acceptable solvents such as lower alcohols, polyols such as glycerol, glycols such as butylene glycol, isoprene glycol, propylene glycol, or polyethylene glycols such as PEG-8, sorbitol, sugars such as glucose, fructose, maltose, lactose or sucrose, and mixtures thereof. Other cosmetically acceptable carriers may include mineral oils, paraffin, silicone oils, synthetic or natural esters, and waxes.

The cosmetically acceptable carrier is typically present in an amount of from about 0.1% to about 90%, from about 0.1% to about 50%, from about 0.1 to about 25%, or from about 0.1% to about 15% by weight of the cosmetic composition. In one embodiment, the amount of cosmetically acceptable carrier is equal to or less than the combined amount of silicone amine and anionic silicone.

The cosmetic compositions can further comprise at least one auxiliary ingredient, including a film forming agent, surfactant, conditioning agent, and skin active agent.

Examples of film forming agents useful in the cosmetic compositions include but are not limited to polysilicone 8, polydimethylsiloxane, dimethylsiloxane/3-thiopropyl methyl siloxane copolymer, vinylpyyrolidone/vinylacetate copolymer, polyvinyacetate, starch, polyquaternium-4, polyquaternium-11, acrylates/steareth-2 methacrylate crosspolymer, vinylacetate/vinyl neodecanoate copolymer, polyester-5, cetyl ethylhexanoate, vinyl acetate, crotonate/vinyl neodecanoate copolymer, 2-acryamido-2-methyl propane sulfonic acid (AMPS)/acrylic acid (AA) copolymer, AMPS/AA/acryl methacrylate copolymer, polyacrylamide, C₁₃-C₁₄ isoparaffin, laureth-7, octylacrylamide, and acrylate/butylaminoethylmethacrylate copolymer.

The film forming agent may be employed in an amount sufficient to impart and/or maintain a shape on the hair. Typically, it will be employed in an amount of from greater than 0 to 30% by weight, preferably from 1 to 10% by weight, and more preferably from 1 to 5% by weight, based on the total weight of the cosmetic composition.

Examples of surfactants that are useful in the cosmetic compositions include but are not limited to amphoteric surfactants such as cocamidoethyl betaine, cocamidopropyl betaine, cocamidopropyl hydroxysultaine, cocamidopropyl dimethylamine propionate, cocoamphoglycinate, cocoamphocarboxypropionate, cocoamphocarboxyglycinate, coco-betaine, cocoamphopropionate, cocoamphopropylsulfonate and mixtures thereof.

Other examples of surfactants that are useful in the cosmetic compositions include but are not limited to sodium lauryl ether sulfate, sodium lauryl sulfate, ammonium lauryl ether sulfate, ammonium lauryl sulfate, disodium laureth sulfosuccinate, and mixtures thereof.

Examples of conditioning agents useful in the cosmetic compositions include but are not limited to polyalkyl siloxanes, polyaryl siloxanes, polyalkylaryl siloxanes, polyether siloxane copolymers, and mixtures thereof. For example, the conditioning agent can be a polydimethyl siloxane, a polydiethyl siloxane, a polydipropyl siloxane, a polymethylethyl siloxane, a polymethylpropylsiloxane, a polymethylphenylsiloxane, and mixtures thereof. In one embodiment, the conditioning agent is polydimethyl siloxane.

The conditioning agent may also be chosen from amino acids, proteins, extracts, fats, oils, esters, hydrocarbons, quats, polyquats, polyols, humectants, fatty acids, and mixtures thereof.

Examples of skin active agents of the present disclosure include skin active agents chosen from hydroxy acids, vitamins, sunscreens, UV filters, humectants, glycols, polyols, self-tanning ingredients, antioxidants and mixtures thereof.

Other examples of skin active agents of the present disclosure include skin active agents chosen from photoprotective agents, self-tanning agents, desquamating agents, depigmenting agents, moisturizing agents, skin lightening agents, anti-aging ingredients, anti-wrinkle agents, anti-dandruff agents and mixtures thereof.

The cosmetic compositions typically comprise from about 0.01% to about 90%, from about 0.05% to about 75%, from about 0.1% to about 50%, or from about 0.5% to about 25% by weight of the at least one auxiliary ingredient based on the total weight of the cosmetic composition.

The cosmetic compositions of the present disclosure can also optionally include an additional component, for instance those chosen from the non-exhaustive list such as shine agents, thickeners and/or oils, viscosity modifiers, rheology-modifying agents, plant extracts, humectants, reducing agents, sequestering agents, softeners, antifoams, emollients, basifying and acidifying agents, gelling agents, wetting agents, spreading agents, dispersants, plasticizers, preservatives, direct dyes or oxidation dyes, colorants, pigments, mineral fillers, clays, colloidal minerals, nacres, nacreous agents, emulsifying agents, fragrances, peptizers, preserving agents, fixing or non-fixing polymers, ceramides, proteins, aliphatic or aromatic alcohols, volatile silicones, hydrocarbons, mineral, organic or plant oils, waxes, paraffins, fatty acids, associative or non-associative thickening polymers, fatty amides, fatty esters, and fatty alcohols.

Examples of shine agents useful in the cosmetic compositions include but are not limited to silicones, alkoxylated silicones, oils, ethoxylated oils, fats, esters, transesters, hydrocarbons, quats and mixtures thereof. Suitable silicone compounds, which are different than the (a) a silicone amine and (b) an anionic silicone include, but are not limited to, Dimethicone, Dimethiconol, Cyclomethicone, Phenyltrimethicone, Aminopropyl Phenyltrimethicone, Trimethyl Pentaphenyl Trisiloxane, Cetyl Dimethicone, Alkyl Dimethicone, and mixtures thereof.

The shine agent may be employed in an amount from about 0.01% to about 95% by weight based on the total weight of the cosmetic composition. Typically, the shine agent is added in an amount from about 1% to about 50% by weight based on the weight of the cosmetic composition and more typically from about 5% to about 20% by weight based on the total weight of the cosmetic composition.

The present disclosure is also directed to the methods of beneficially coating keratinous substrates such as hair, scalp, skin, lips, nails, and eyelashes comprising applying the cosmetic compositions of the instant disclosure to the keratinous substrates. For example, the cosmetic compositions can be used for (1) imparting shine to hair; (2) improving the condition/manageability of hair; (3) preventing hair from becoming frizzy, i.e., as an “anti-frizz” agent; (4) styling hair; (5) color retention or improving fade resistance of artificially colored hair, (6) moisture retention on skin, scalp or hair; (7) providing sun protection; and (8) providing skin care benefits such as, for example, anti-aging, whitening/depigmentation, anti-acne, etc.

### DETAILED DESCRIPTION

Where the following terms are used in this specification, they are used as defined below.

Other than in the operating examples, or where otherwise indicated, all numbers expressing quantities of ingredients and/or reaction conditions are to be understood as being modified in all instances by the term “about”.

The terms “comprising,” “having,” and “including” are used in their open, non-limiting sense.

The terms “a” and “the” are understood to encompass the plural as well as the singular.

The term “about” when referring to a value, is meant specifically that a measurement can be rounded to the value using a standard convention for rounding numbers. For example, “about 1.5” is 1.45 to 1.54.

As used herein, the expression “at least one” means one or more and thus includes individual components as well as mixtures/combinations.

“Cosmetically acceptable” means that the item in question is compatible with any keratinous substrate. For example, “cosmetically acceptable carrier” means a carrier that is compatible with any keratinous substrate.

“Keratinous substrate” includes, for example, skin, hair, nails, eyelashes, eyelids, eyebrows, lips and any other area of body or facial skin.

“Film former” or “film forming agent” as used herein means a polymer or resin that leaves a film on the substrate to which it is applied, for example, after a solvent accompanying the film former has evaporated, absorbed into and/or dissipated on the substrate.

As used herein, the term “beneficially” includes, for example, providing benefits to keratinous substrates such as shine to hair, condition/manageability of hair, anti-frizz to hair, styling to hair, color retention/fade resistance to artificially colored hair, moisture retention to skin, scalp or hair; sun protection; and skin care benefits such as, for example, anti-aging, whitening/depigmentation, anti-acne, etc.

### Silicone Amines

The term “silicone amine” denotes any silicone polymer that is a base that comprises at least two primary, secondary or tertiary amine groups. The multiple amine groups can be the same or different. The term “silicone amine” does not include cationic alkyl quaternary silicone compounds (e.g., polyquaterniums), which have a positive charge due to nitrogen being substituted with four alkyl groups.

The silicone amine typically has an amine number of 0.4 meq/g or greater, from 0.5 to 5 meq/g, or from 0.5 to 3.5 meq/g. The amine number is the number of amine milliequivalents per gram of compound. This number can be determined by methods conventional in the art, for example by titration with a colored indicator or by potentiometric titration.

In one embodiment, the silicone amine corresponds to the following formula:

R′ₐG₃₋ₐ-Si(OSiG₂)ₙ-(OSiG_{b}R′_{2-b})ₘ—O—SiG₃₋ₐ-R′ₐ

wherein,

G is chosen from a hydrogen atom, phenyl, OH and a C₁-C₈ alkyl group, for example a methyl group;

a is chosen from the number 0 and an integer from 1 to 3;

b is chosen from 0 and 1;

m and “n” are numbers such that the sum (n+m) is from 1 to 2000, 50 to 150, for example, n can be a number from 0 to 1999 or from 49 to 149 and m can be a number from 1 to 2000 or from 1 to 10;

R′ is chosen from monovalent radicals of formula —C_{q}H_{2q}L, where q is chosen from a number from 2 to 8 and L is chosen from:

—N(R″)—CH₂—CH₂—N(R″)₂,

—N(R″)₂,

—N⁺H(R″)₂A⁻,

—N⁺H₂(R″)A⁻, and

—N(R″)—CH₂—CH₂—N+R″H₂A⁻,

wherein R″, which can be identical or different, is selected from a hydrogen atom, phenyl, benzyl and saturated monovalent hydrocarbon-comprising radicals, for example, an alkyl radical having from 1 to 20 carbon atoms, a methyl radical; and

A⁻ is chosen from organic and inorganic anions, for example a halide ion, such as, for example, fluoride, chloride, bromide or iodide.

Products corresponding to the group of silicone amines are in particular amodimethicone, trimethylsilylamodimethicone, aminopropyl phenyltrimethicone, PEG-7 amodimethicone, aminopropyl triethyoxysilane, alkyl modified amino silicone, amino silanes, PEG-8 amodimethicone, bis-isobutyl PEG-14/amodimethicone copolymer, bis-isobutyl PEG-15/amodimethicone copolymer, bis-isobutyl PEG/PPG-20/35/amodimethicone copolymer, Trideceth-9 PG-Amodimethicone and mixtures thereof.

In one embodiment, the silicone amine is trimethylsilylamodimethicone (International Cosmetic Ingredient Dictionary and Handbook, Seventh Edition, 1997), corresponding to the formula:

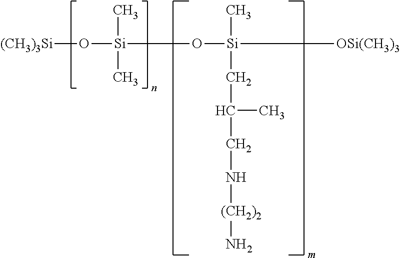

in which m and n are numbers such that the sum (n+m) is from 1 to 2000, 50 to 150, for example, n can be a number from 0 to 1999 or from 49 to 149 and m can be a number from 1 to 2000 or from 1 to 10.

In another embodiment, the silicone amine is amodimethicone (International Cosmetic Ingredient Dictionary and Handbook, Seventh Edition, 1997), corresponding to the formula:

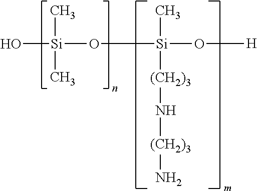

in which m and n are numbers such that the sum (n+m) is from 1 to 2000, 50 to 150, for example, n can be a number from 0 to 1999 or from 49 to 149 and m can be a number from 1 to 2000 or from 1 to 10. Such polymers are disclosed, for example, in Patent Application EP-A-95,238, the disclosure of which is herein incorporated by reference in its entirety.

In yet another embodiment, the silicone amine chosen from polyethylene glycol derivatives of amodimethicone such as PEG-7 amodimethicone and PEG-8 amodimethicone and modified aminosiloxanes such as Trideceth-9 PG-Amodimethicone (commercially available as SilCare Silicone SEA sold by Clariant International, LTD).

The cosmetic compositions can comprise from about 1% to about 90%, from about 1% to about 75%, from about 1% to about 50%, from about 1% to about 25%, or from about 1% to about 15% by weight of the silicone amine based on the total weight of the cosmetic composition. The cosmetic composition can comprise silicone amine in the amount of about 95%, 90%, 80%, 70%, 60%, 50%, 40%, 30%, 20%, 10%, 5% or less based on the total weight of the cosmetic composition. The cosmetic composition can comprise silicone amine in the amount of about 5%, 10%, 20%, 30%, 40%, 50%, 60%, 70%, 80%, 90%, 95%, or more based on the total weight of the cosmetic composition. Additionally the amount of silicone amine can be any combination of the above values, for example from about 20% to about 95% or from about 50% to about 60% by weight based on the total weight of the cosmetic compositions.

### Anionic Silicones

In general, non-limiting examples of anionic silicones include silicone carboxylates, silicone phosphates, silicone sulfates, silicone sulfosuccinates, silicone sulfonates, and mixtures thereof.

Suitable silicone carboxylates may be chosen from water soluble silicone compounds comprising at least one carboxylic acid group, oil soluble silicone compounds comprising at least one carboxylic acid group, water-dispersible silicone compounds comprising at least one carboxylic acid group, and silicone compounds comprising at least one carboxylic acid group which are soluble in organic solvents. In one embodiment, the silicone carboxylate further comprises at least one alkoxylated chain, wherein the at least one alkoxy group may be chosen from terminal alkoxy groups, pendant alkoxy groups, and alkoxy groups which are intercalated in the skeleton of the at least one silicone compound. Non-limiting examples of at least one alkoxy group include ethylene oxide groups and propylene oxide groups.

The at least one carboxylic acid group may be chosen from terminal carboxylic acid groups and pendant carboxylic acid groups. Further, the at least one carboxylic acid may be chosen from carboxylic acid groups in free acid form, i.e., —COOH, and carboxylic acid groups in salt form, i.e., —COOM, wherein M may be chosen from inorganic cations, such as, for example, potassium cations and sodium cations, and organic cations.

In one embodiment, the silicone carboxylate is a compound of formula (I):

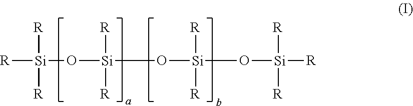

wherein:

a is an integer ranging from 1 to 100;

b is an integer ranging from 0 to 500;

R, which may be identical or different, are each chosen from optionally substituted hydrocarbon groups comprising from 1 to 9 carbon atoms, optionally substituted phenyl groups, and groups of formula (II):

—(CH₂)₃—O-(EO)_{c}—(PO)_{d}-(EO)ₑ—C(O)—R′—C(O)—OH (II)

wherein:

c, d, and e, which may be identical or different, are each integers ranging from 0 to 20;

EO is an ethylene oxide group;

PO is a propylene oxide group; and

R″ is chosen from optionally substituted divalent hydrocarbons, such as alkylene groups and alkenylene groups comprising from 2 to 22 carbon atoms, and optionally substituted divalent aromatic groups, such as groups of formula (III):

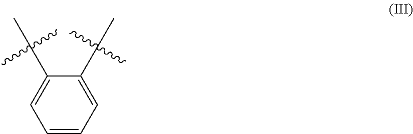 [SMILES: *c1ccccc1*]

and groups of formula (IV):

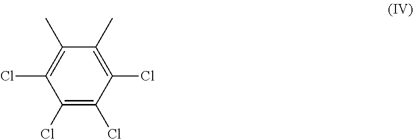

with the proviso that at least one of the R groups is chosen from groups of formula (II) and with the further proviso that when only one of the R groups is chosen from groups of formula (II), the other R groups are not all methyl groups.

Non-limiting examples of silicone carboxylates include those commercially available from Noveon under the name Ultrasil® CA-1 Silicone (Dimethicone PEG-7 Phthalate) and Ultrasil® CA-2 Silicone (Dimethicone PEG-7 Succinate), both of which correspond to formula (V) below. Thus, in one embodiment, the at least one silicone carboxylate is chosen from a compound of formula (V) and salts thereof:

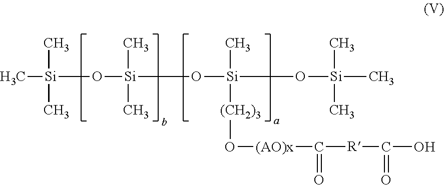

wherein:

a is an integer ranging from 1 to 100;

b is an integer ranging from 0 to 500;

AO is chosen from groups of formula (VI):

-(EO)c—(PO)d-(EO)e— (VI)

wherein:

c, d, and e, which may be identical or different, are each integers ranging from 0 to 20; EO is an ethylene oxide group;

PO is a propylene oxide group;

x is an integer ranging from 0 to 60;

R″ is chosen from optionally substituted divalent hydrocarbons, such as alkylene groups and alkenylene groups comprising from 2 to 22 carbon atoms, and optionally substituted divalent aromatic groups, such as groups of formula (III):

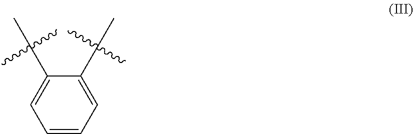 [SMILES: *c1ccccc1*]

and groups of formula (IV):

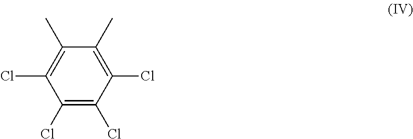

Non-limiting examples of silicone carboxylates include those described in U.S. Pat. Nos. 5,248,783 and 5,739,371, the disclosures of which are incorporated herein by reference, and which are silicone compounds of formula (I).

Suitable silicone phosphates may be chosen from water-soluble silicone compounds comprising at least one phosphate group, oil soluble silicone compounds comprising at least one phosphate group, water-dispersible silicone compounds comprising at least one phosphate group, and silicone compounds comprising at least one phosphate group.

In one embodiment, the silicone phosphate comprises at least one alkoxylated chain, wherein the at least one alkoxy group may be chosen from terminal alkoxy groups, pendant alkoxy groups, and alkoxy groups which are intercalated in the skeleton of the at least one silicone compound. Non-limiting examples of at least one alkoxy group include ethylene oxide groups (“EO”=—CH₂—CH₂—O—) and propylene oxide groups (“PO”═C₃H₆O).

The at least one phosphate group may be chosen from terminal phosphate groups and pendant phosphate groups. Further, the at least one phosphate group may be chosen from groups of formula —O—P(O) (OH)₂, groups of formula —O—P(O) (OH)(OR), and groups of formula —O—P(O)(OR)₂, wherein R may be chosen from H, inorganic cations, and organic cations. Non-limiting examples of inorganic cations include alkali metals, such as, for example, potassium lithium, and sodium. A non-limiting example of organic cations is at least one additional silicone compound which may be identical to or different from the at least one silicone compound bonded to the other oxygen of the phosphate group.

In one embodiment, the silicone phosphate is a compound of formula (VII):

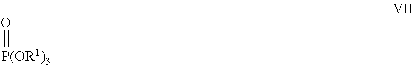

wherein R₁, which may be identical or different, is chosen from H, organic cations, inorganic cations, optionally substituted hydrocarbons (such as alkyl groups and alkenyl groups comprising from 1 to 22 carbon atoms), and optionally substituted aromatic groups.

In one embodiment, the silicone phosphate is a compound of formula (VIII) and salts thereof:

CH₃(CH₂)ₓ—O-(EO)_{c}—(PO)_{d}-(EO)ₑ—CH₂CH₂— (VIII)

wherein:

c, and d, which may be identical or different, are each integers ranging from 0 to 20;

e is an integer ranging from 0 to 19; and

x is an integer ranging from 0 to 21.

In another embodiment, the silicone phosphate is a compound of formula (IX):

HO-(EO)_{c}—(PO)_{d}-(EO)ₑ—(CH₂)ₓ— (IX)

wherein:

c, d, and e, which may be identical or different, are each integers ranging from 0 to 20;

and x is an integer ranging from 0 to 21.

In one embodiment, the silicone phosphate is a compound of formula (X):

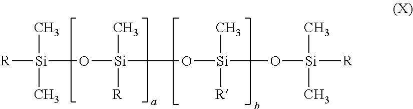

wherein a is an integer ranging from 0 to 200;

b is an integer ranging from 0 to 200;

R′, which may be identical or different, are each chosen from optionally substituted hydrocarbons, such as alkyl groups and alkenyl groups comprising from 1 to 22 carbon atoms, optionally substituted aromatic groups, groups of formula (III) as defined above and salts thereof; and

R, which may be identical or different, are each chosen from optionally substituted hydrocarbons, such as alkyl groups and alkenyl groups comprising from 1 to 22 carbon atoms, optionally substituted aromatic groups, optionally substituted divalent hydrocarbons, such as alkylene groups and alkenylene groups comprising from 1 to 22 carbon atoms, optionally substituted divalent aromatic groups, groups of formula (VIII) as defined above and salts thereof.

In one embodiment, the silicone phosphate is a compound of formula (XI):

-(EO)_{c}—(PO)_{d}-(EO)ₑ—(CH₂)₃— (XI)

wherein:

the (CH₂)₃ end is bonded to the silicone of the compound of formula (X) and the (EO) or (PO) end, if present, is bonded to the oxygen of the compound of formula (I);

c, d, and e, which may be identical or different, are each integers ranging from 0 to 20; EO is an ethylene oxide group; and

PO is a propylene oxide group; and with the proviso that at least one R is chosen from groups of formula (XI) and salts thereof; and with the further proviso that at least one R₁ is chosen from groups of formula (X) and salts thereof and at least one other R₁ is chosen from H, organic cations, and inorganic cations.

Non-limiting examples of inorganic cations that form salt with the above compounds include alkali metals, such as potassium, lithium, and sodium.

Non-limiting examples of silicone phosphates include dimethicone PEG-7 phosphate, dimethicone PEG-8 phosphate, dimethicone PEG-10 phosphate, potassium dimethicone PEG-7 panthenyl phosphate (PECOSIL® SPP-50), dimethicone PEG/PPG-7/4 phosphate, dimethicone PEG/PPG-12/4 phosphate, and mixtures thereof. In some aspects, the (a) poly-silicone phosphate is dimethicone PEG-8 phosphate.

Silicone phosphates are commercially available from Phoenix Chemical, Inc. of New Jersey under the name of Pecosil®, such as Pecosil® PS-100, Pecosil® PS-112, Pecosil® PS-150, Pecosil® PS-200, Pecosil® WDS-100, Pecosil® WDS-200, Pecosil® PS-100 B, and Pecosil® PS-100 K and from Siltech under the name Silphos A-100 and Silphos A-150.

Non-limiting examples of silicone phosphates include dimethicone PEG-7 phosphate, dimethicone PEG-8 phosphate, dimethicone PEG-10 phosphate, potassium dimethicone PEG-7 panthenyl phosphate (PECOSIL® SPP-50), dimethicone PEG/PPG-7/4 phosphate, dimethicone PEG/PPG-12/4 phosphate, and mixtures thereof. In some aspects, the (a) poly-silicone phosphate is dimethicone PEG-8 phosphate.

Silicone phosphates are commercially available from Phoenix Chemical, Inc. of New Jersey under the name of Pecosil®, such as Pecosil® PS-100, Pecosil® PS-112, Pecosil® PS-150, Pecosil® PS-200, Pecosil® WDS-100, Pecosil® WDS-200, Pecosil® PS-100 B, and Pecosil® PS-100 K and from Siltech under the name Silphos A-100 and Silphos A-150.

Suitable silicone sulfates for use in the present invention include those represented by formula XII:

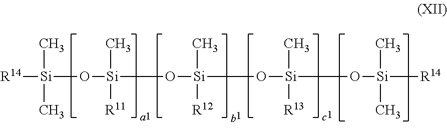

wherein R¹¹ is selected from lower alkyl having one to eight carbon atoms or phenyl, R¹² is —(CH₂)₃—O-(EO)ₓ—(PO)_{y}-(EO)_{z}—SO₃³¹-M⁺ wherein M is a cation and is selected from Na, K, Li, or NH₄; x, y and z are integers independently ranging from 0 to 100; R¹³ is —(CH₂)—O-(EO)ₓ—(PO)_{y}-(EO)_{z}—H; R¹⁴ is methyl or hydroxyl; a¹ and c¹ are independently integers ranging from 0 to 50; b¹ is an integer ranging from 1 to 50. An example thereof is Ultrasil SA-1 silicone (dimethicone PEG-7 phthalate) commercially available from Noveon.

Suitable silicone sulfosuccinates which may be employed include, but are not limited to, those corresponding to formula XIII:

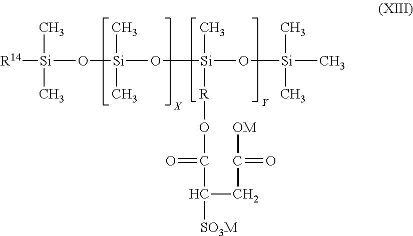

wherein R represents a divalent radical selected from

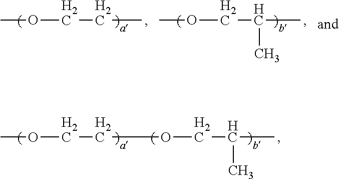

wherein a′ and b′ range from 0 to 30; x and y are such that the molecular weight ranges from 700 to 1600, and M is an alkali metal such as sodium or potassium, or an ammonium group.

Other non-limiting examples of the at least one silicone compound include those described in U.S. Pat. Nos. 5,070,171, 5,093,452, and 5,149,765 the disclosures of which are incorporated herein by reference in their entirety.

The cosmetic compositions typically comprise from about 1% to about 90%, from about 1% to about 75%, from about 1% to about 50%, from about 1% to about 25% or from about 1% to about 15% by weight of the anionic silicone based on the total weight of the cosmetic composition. The cosmetic composition can comprise anionic silicone in the amount of about 95%, 90%, 80%, 70%, 60%, 50%, 40%, 30%, 20%, 10%, 5% or less based on the total weight of the cosmetic composition. The cosmetic composition can comprise anionic in the amount of about 5%, 10%, 20%, 30%, 40%, 50%, 60%, 70%, 80%, 90%, 95%, or more based on the total weight of the cosmetic composition. Additionally the amount of anionic silicone can be any combination of the above values, for example from about 20% to about 95%, or from about 50% to about 60% by weight based on the total weight of the cosmetic compositions.

In one embodiment, the ratio of silicone amine to anionic silicone is from about 1:20 to about 20:1, from about 1:10 to about 10:1, from about 5:1 to about 1:5, from about 3:1 to about 1:3, about 2:1, or about 1:1.

Without intending to be bound by theory, it is believed that the association or combination of silicone amine and anionic silicone results in a silicone amine-anionic silicone complex which allows compositions containing the complex to provide various cosmetic and dermatological benefits to keratinous substrates such as styling, anti-frizz, conditioning, shine, color retention, moisture retention, sun protection and skin care benefits.

### Colorants

The colorants of the present disclosure may be chosen from oxidative dyes, direct dyes, pigments, natural colorants, and mixtures thereof. Other suitable hair colorants include, but are not limited to, liposoluble dyes, nacreous pigments, pearling agents, leuco dyes, optical lightening colorants, and optically-variable pigments.

Oxidative Dye

The oxidative dye of the present disclosure may be selected from any type of oxidative dye useful for imparting color to hair. The oxidative dye may also encompass a wide variety of oxidation dye precursors. These include primary dye intermediates and couplers.

Primary Dye Intermediates

Examples of primary dye intermediates include ortho or para aminophenols, ortho or para phenylenediamines, double bases, heterocyclic bases, and the acid addition salts thereof.

The para-phenylenediamines which can be used include compounds of the following formula (XIV) and their addition salts with an acid:

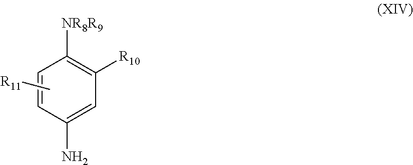

in which:

R₈ represents a hydrogen atom, a C₁-C₄ alkyl radical, a C₁-C₄ monohydroxyalkyl radical, a C₂-C₄ polyhydroxyalkyl radical, a (C₁-C₄)alkoxy(C₁-C₄)alkyl radical, a C₁-C₄ alkyl radical substituted by a nitrogenous group, a phenyl radical or a 4′-aminophenyl radical;

R₉ represents a hydrogen atom, a C₁-C₄ alkyl radical, a C₁-C₄ monohydroxyalkyl radical, a C₂-C₄ polyhydroxyalkyl radical, a (C₁-C₄)alkoxy(C₁-C₄)alkyl radical or a C₁-C₄ radical substituted by a nitrogenous group;

R₈ and R₉ can also form, with the nitrogen atom which carries them, a 5- or 6-membered nitrogenous heterocycle optionally substituted by one or more alkyl, hydroxyl or ureido groups;

R₁₀ represents a hydrogen atom, a halogen atom, such as a chlorine atom, a C₁-C₄ alkyl radical, a sulpho radical, a carboxyl radical, a C₁-C₄ monohydroxyalkyl radical, a C₁-C₄ hydroxyalkoxy radical, a C₁-C₄ acetylaminoalkoxy radical, a C₁-C₄ mesylaminoalkoxy radical or C₁-C₄ carbamoylaminoalkoxy radicals;

R₁₁ represents a hydrogen atom, a halogen atom or a C₁-C₄ alkyl radical.

The nitrogenous groups in the above formula (XIV) include amino, mono(C₁-C₄)alkylamino, di(C₁-C₄)alkylamino, tri(C₁-C₄)alkylamino, monohydroxy(C₁-C₄)alkylamino, imidazolinium and ammonium radicals.

The para-phenylenediamines of above formula (XIV) include para-phenylenediamine, para-toluoylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-(β-hydroxyethyl)-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-(β-hydroxyethyloxy)-para-phenylenediamine, 2-(β-acetylaminoethyloxy)-para-phenylenediamine, N-(β-methoxyethyl)-para-phenylene-diamine, 2-methyl-1-N-(β-hydroxyethyl)-para-phenylenediamine and their addition salts with an acid.

In one embodiment, the para-phenylenediamines of above formula (XIV) include para-phenylenediamine, para-toluoylenediamine, 2-isopropyl-para-phenylenediamine, 2-(β-hydroxyethyl)-para-phenylenediamine, 2-(β-hydroxyethyloxy)-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and their addition salts with an acid.

The ortho-phenylenediamines include N1-(2-hydroxyethyl)-4-nitro-o-phenylenediamine, 4-methyl-o-phenylenediamine, and 4-nitro-o-phenylenediamine and acid addition salts thereof.

As used herein, the term “double bases” means compounds comprising at least two aromatic nuclei having at least one of amino and hydroxyl groups.

Examples include compounds corresponding to the following formula (XV) and their addition salts with an acid:

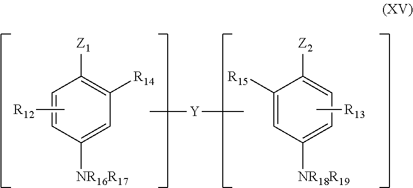

in which:

Z₁ and Z₂, which are identical or different, represent a hydroxyl or —NH₂ radical which can be substituted by a C₁-C₄ alkyl radical or by a connecting arm Y;

the connecting arm Y represents a linear or branched alkylene chain comprising from 1 to 14 carbon atoms which can be interrupted or terminated by one or more nitrogenous groups and/or by one or more heteroatoms, such as oxygen, sulphur or nitrogen atoms, and which is optionally substituted by one or more hydroxyl or C₁-C₆ alkoxy radicals;

R₁₂ and R₁₃ represent a hydrogen or halogen atom, a C₁-C₄ alkyl radical, a C₁-C₄ monohydroxyalkyl radical, a C₂-C₄ polyhydroxyalkyl radical, a C₁-C₄ aminoalkyl radical or a connecting arm Y;
R₁₄, R₁₅, R₁₆, R₁₇, R₁₈ and R₁₉, which are identical or different, represent a hydrogen atom, a connecting arm Y or a C₁-C₄ alkyl radical;

it being understood that the compounds of formula (XV) only comprise a single connecting arm Y per molecule.

Nitrogenous groups of the above formula (XV) include amino, mono(C1-C4)alkylamino, di(C1-C4)alkylamino, tri(C1-C4)alkylamino, monohydroxy(C1-C4)alkylamino, imidazolinium and ammonium radicals.

Additional examples of double bases of above formula (XV) include of N,N′-bis(β-hydroxyethyl)-N,N′-bis(4′-aminophenyl)-1,3-diamino-propanol, N,N′-bis(β-hydroxyethyl)-N,N′-bis(4′-aminophenyl)ethylenediamine, N,N′-bis(4-aminophenyl)-tetramethylenediamine, N,N′-bis(β-hydroxyethyl)-N,N′-bis(4-amino-phenyl)tetramethylenediamine, N,N′-bis(4-methylaminophenyl)tetramethylenediamine, N,N′-diethyl-N,N′-bis(4′-amino-3′-methylphenyl)ethylenediamine, 1,8-bis(2,5-diaminophenoxy)-3,5-dioxaoctane and their addition salts with an acid.

In one embodiment the double base is N,N′-Bis(β-hydroxyethyl)-N,N′-bis(4′-aminophenyl)-1,3-diaminopropanol, 1,8-bis(2,5-diaminophenoxy)-3,5-dioxaoctane or one of their addition salts with an acid.

The para-aminophenols which can be used include compounds of the following formula (XVI) and their addition salts with an acid:

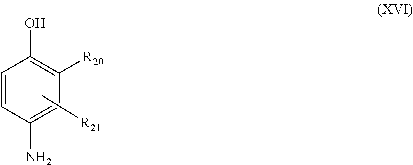

in which:
R₂₀ represents a hydrogen atom, a halogen atom, such as fluorine, a C₁-C₄ alkyl radical, a C₁-C₄ monohydroxyalkyl radical, a (C₁-C₄)alkoxy(C₁-C₄) alkyl radical, a C₁-C₄ aminoalkyl radical or a hydroxy(C₁-C₄)alkylamino-(C₁-C₄)alkyl radical,

R₂₁ represents a hydrogen atom, a halogen atom, such as fluorine, a C₁-C₄ alkyl radical, a C₁-C₄ monohydroxyalkyl radical, a C₂-C₄ polyhydroxyalkyl radical, a C₁-C₄ aminoalkyl radical, a C₁-C₄ cyanoalkyl radical or a (C₁-C₄)alkoxy(C₁-C₄)alkyl radical.

Among the para-aminophenols, mention may be made of para-aminophenol, 4-amino-3-methylphenol, 4-amino-3-fluorophenol, 4-amino-3-hydroxymethylphenol, 4-amino-2-methylphenol, 4-amino-2-hydroxymethyl phenol, 4-amino-2-methoxymethylphenol, 4-amino-2-aminomethylphenol, 4-amino-2-(β-hydroxyethylaminomethyl)phenol, N-methyl-para-aminophenol, and the acid addition salts thereof.

The ortho-aminophenols that may be used as oxidation bases in the context of certain embodiments may be chosen from 2-aminophenol, 2-amino-1-hydroxy-5-methylbenzene, 2-amino-1-hydroxy-6-methylbenzene, 5-acetamido-2-aminophenol, and the acid addition salts thereof.

Heterocyclic bases that can be used as oxidation bases in the methods of coloring keratinous fibers include pyridine derivatives, pyrimidine derivatives, pyrazole derivatives, pyrazolinone derivatives, and the acid addition salts thereof.

Pyridine derivatives include the compounds described, for example, in patents GB 1,026,978 and GB 1,153,196, as well as the compounds 2,5-diaminopyridine, 2-(4-methoxyphenyl)amino-3-aminopyridine, 2,3-diamino-6-methoxypyridine, 2-(β-methoxyethyl)amino-3-amino-6 methoxypyridine, 3,4-diaminopyridine, and the acid addition salts thereof.

Pyrimidine derivatives include the compounds disclosed, for example, in German Patent DE 2 359 399 or Japanese Patents JP 88-169 571 and JP 91-10659 or Patent Application WO 96/15765, such as 2,4,5,6-tetra-aminopyrimidine, 4-hydroxy-2,5,6-triaminopyrimidine, 2-hydroxy-4,5,6-triamino-pyrimidine, 2,4-dihydroxy-5,6-diaminopyrimidine, 2,5,6-triaminopyrimidine, and the pyrazolopyrimidine derivatives, such as those mentioned in French Application FR-A-2 750 048 and among which may be mentioned pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol; 2-(7-aminopyrazolo[1,5-a]pyrimidin-3-ylamino)ethanol; 2-[(3-aminopyrazolo[1,5-a]pyrimidin-7-yl)(2-hydroxyethyl)amino]ethanol; 2-[(7-aminopyrazolo[1,5-a]pyrimidin-3-yl)(2-hydroxyethyl)amino]ethanol; 5,6-dimethyl-pyrazolo[1,5-a]pyrimidine-3,7-diamine; 2,6-dimethylpyrazolo[1,5-a]pyrimidine-3,7-diamine; 2,5,N7,N7-tetramethylpyrazolo[1,5-a]pyrimidine-3,7-diamine; 3-amino-5-methyl-7-(imidazolylpropylamino)pyrazolo[1,5-a]pyrimidine; and their addition salts and their tautomeric forms, when there exists a tautomeric equilibrium, and their addition salts with an acid.

Pyrazole and pyrazolinone derivatives include the compounds described in patents DE 3,843,892, DE 4,133,957 and patent applications WO 94/08969, WO 94/08970, FR-A-2,733,749, and DE 195 43 988, such as 4,5-diamino-1-methyl-pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-(β-hydroxyethyl)pyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethyl-pyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, 2-(4,5-diamino-1H-pyrazol-1-yl), H₂SO₄, 2,3-diamino-6,7-dihydro-1H,5H-pyrazolo[1,2-z]pyrazol-1-one, 1-methyl-3-phenyl-2-pyrazolinone, and the acid addition salts thereof.

Primary intermediates include p-phenylenediamine, p-aminophenol, o-aminophenol, N,N-bis(β-hydroxyethyl)-p-phenylenediamine, 2,5-diaminotoluene, their salts and mixtures thereof.

The primary intermediates may be employed in amounts ranging from 0.0001% to 12% by weight, or from 0.0001% to 8.0% by weight, or, from 0.005% to 5% by weight, based on the total weight of the coloring composition.

Color Couplers

The compositions of the present disclosure may also contain coupler compounds. The couplers that may be used in the dyeing method disclosed herein include those conventionally used in oxidative methods of coloring keratinous fibers, for example, meta-aminophenols, meta-phenylenediamines and meta-diphenols, naphthols, mono- or polyhydroxylated naphthalene derivatives, and heterocyclic couplers such as, for example, indole derivatives, indoline derivatives, sesamol and its derivatives, pyridine derivatives, pyrazolotriazole derivatives, pyrazolones, indazoles, benzimidazoles, benzothiazoles, benzoxazoles, 1,3-benzodioxoles, quinolines, benzomorpholine derivatives, pyrazoloazole derivatives, pyrroloazole derivatives, imidazoloazole derivatives, pyrazolopyrimidine derivatives, pyrazoline-3,5-dione derivatives, pyrrolo[3,2-d]oxazole derivatives, pyrazolo[3,4-d]thiazole derivatives, thiazoloazole S-oxide derivatives, thiazoloazole S,S-dioxide derivatives, and the acid addition salts thereof.

Suitable color couplers include, for example, those having the general formula (XVII):

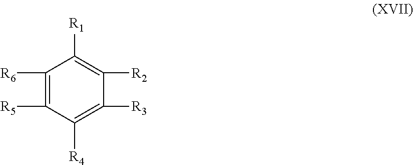

wherein R₁ is unsubstituted hydroxy or amino, or hydroxy or amino substituted with one or more C₁₋₆ hydroxyalkyl groups, R₃ and R₅ are each independently hydrogen, hydroxy, amino, or amino substituted with C₁₋₆ alkyl, C₁₋₆ alkoxy, or C₁₋₆ hydroxyalkyl group; and R₂, R₄, and R₆ are each independently hydrogen, C₁₋₆ alkoxy, C₁₋₆ hydroxyalkyl, or C₁₋₆ alkyl, or R₃ and R₄ together may form a methylenedioxy or ethylenedioxy group. Examples of such compounds include meta-derivatives such as phenols, meta-aminophenols, meta-phenylenediamines, and the like, which may be unsubstituted, or substituted on the amino group or benzene ring with alkyl, hydroxyalkyl, alkylamino groups, and the like. Suitable couplers include m-aminophenol, 2,4-diaminotoluene, 4-amino, 2-hydroxytoluene, phenyl methylpyrazolone, 3,4-methylenedioxyphenol, 3,4-methylenedioxy-1-[(beta-hydroxyethyl)amino]benzene, 1-methoxy-2-amino-4-[(beta-hydroxyethyl)amino]benzene, 1-hydroxy-3-(dimethylamino)benzene, 6-methyl-1-hydroxy-3[(beta-hydroxyethyl)amino]benzene, 2,4-dichloro-1-hydroxy-3-aminobenzene, 1-hydroxy-3-(diethylamino)benzene, 1-hydroxy-2-methyl-3-aminobenzene, 2-chloro-6-methyl-1-hydroxy-3-aminobenzene, 1,3-diaminobenzene, 6-methoxy-1,3-diaminobenzene, 6-hydroxyethoxy-1,3-diaminobenzene, 6-methoxy-5-ethyl-1,3-diaminobenzene, 6-ethoxy-1,3-diaminobenzene, 1-bis(beta-hydroxyethyl)amino-3-aminobenzene, 2-methyl-1,3-diaminobenzene, 6-methoxy-1-amino-3-[(beta-hydroxyethyl)amino]-benzene, 6-(beta-aminoethoxy)-1,3-diaminobenzene, 6-(beta-hydroxyethoxy)-1-amino-3-(methylamino)benzene, 6-carboxymethoxy-1,3-diaminobenzene, 6-ethoxy-1-bis(beta-hydroxyethyl)amino-3-aminobenzene, 6-hydroxyethyl-1,3-diaminobenzene, 1-hydroxy-2-isopropyl-5-methylbenzene, 1,3-dihydroxybenzene, 2-chloro-1,3-dihydroxybenzene, 2-methyl-1,3-dihydroxybenzene, 4-chloro-1,3-dihydroxybenzene, 5,6-dichloro-2-methyl-1,3-dihydroxybenzene, 1-hydroxy-3-amino-benzene, 1-hydroxy-3-(carbamoylmethylamino)benzene, 6-hydroxybenzomorpholine, 4-methyl-2,6-dihydroxypyridine, 2,6-dihydroxypyridine, 2,6-diaminopyridine, 6-aminobenzomorpholine, 1-phenyl-3-methyl-5-pyrazolone, 1-hydroxynaphthalene, 1,7-dihydroxynaphthalene, 1,5-dihydroxynaphthalene, 5-amino-2-methyl phenol, 4-hydroxyindole, 4-hydroxyindoline, 6-hydroxyindole, 6-hydroxyindoline, 2,4-diamionphenoxyethanol, and mixtures thereof.

Other couplers may be chosen, for example, from 2,4-diamino-1-(β-hydroxyethyloxy)benzene, 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino 1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, α-naphthol, 6-hydroxyindole, 4-hydroxyindole, 4-hydroxy-N-methylindole, 6-hydroxyindoline, 2,6-dihydroxy-4-methylpyridine, 1H-3-methylpyrazol-5-one, 1-phenyl-3-methylpyrazol-5-one, 2-amino-3-hydroxypyridine, 3,6-dimethylpyrazolo[3,2-c]-1,2,4-triazole, 2,6-dimethylpyrazolo[1,5-b]-1,2,4-triazole, 6-methylpyrazolo[1,5-a]-benzimidazole, and the acid addition salts thereof.

In one embodiment, the couplers include resorcinol, 1-naphthol, 2-methylresorcinol, 4-amino-2-hydroxy toluene, m-aminophenol, 2,4-diaminophenoxyethanol, phenyl methylpyrazolone, hydroxybenzomorpholine, 2-methyl-5-hydroxyetyylaminophenol, 6-hydroxyindole, 2-amino-3-hydroxypyridine, 5-amino-6-chloro-o-cresol, 4-chlororesorcinol, their salts, and mixtures thereof.

When they are present, couplers may be present in amounts ranging from 0.0001% to 12% by weight; or from 0.001% to 8% by weight, based on the total weight of the coloring composition.

In general, the acid addition salts of the oxidation bases and couplers may be chosen from hydrochlorides, hydrobromides, sulphates, tartrates, lactates, and acetates.

Oxidizing Agent

The compositions of the present disclosure may require an oxidizing agent when the colorant comprises an oxidative dye. Oxidizing agents are used in an amount sufficient for the oxidative dye to develop a color. The oxidizing agents may be, for example, peroxide, a persulfate, a perborate, a percarbonate, alkali metal bromates, ferricyanides or a mixture thereof. Oxidizing agents that may also be used include at least one redox enzyme such as laccases, peroxidases, and 2-electron oxidoreductases, such as uricase, where appropriate in the presence of their respective donor or co-factor.

In one embodiment, the oxidizing agent is hydrogen peroxide present in an aqueous solution whose titre may range from 1 to 40 volumes, such as from 5 to 40 volumes.

In another embodiment, the oxidizing agent is a persulfate and/or a monopersulfate such as, for example, potassium persulfate, sodium persulfate, ammonium persulfate, as well as mixtures thereof. In one embodiment the oxidizing agents in the present disclosure are selected from hydrogen peroxide, potassium persulfate, sodium persulfate and mixtures thereof.

In general, the oxidizing agent will be present in an amount of at least 1% by weight, based on the total weight of the coloring composition.

Typically, the oxidizing agent is provided in the form of a developer composition.

In one embodiment, the oxidizing agent is present in an amount of at least 1% by weight, based on the total weight of the developer composition.

In another embodiment, the oxidizing agent is present in an amount ranging from 1% by weight to 80% by weight, or from 5% by weight to 75% by weight, based on the total weight of the developer composition.

In one particular embodiment, the developer composition is aqueous or is in the form of an emulsion.

In another embodiment, the developer composition is substantially anhydrous.

The term “substantially anhydrous” means that the developer is either completely free of water or contains no appreciable amount of water, for example, no more than 1% by weight, or no more than 0.5% by weight, based on the weight of the developer composition.

The developer composition can contain at least one solvent, chosen from water, organic solvents, and mixtures thereof.

When the developer composition is substantially anhydrous, developer composition may comprise at least one solvent chosen from organic solvents.

Suitable organic solvents for use in the developer composition include ethanol, isopropyl alcohol, benzyl alcohol, phenyl ethyl alcohol, glycols and glycol ethers, such as propylene glycol, hexylene glycol, ethylene glycol monomethyl, monoethyl or monobutyl ether, propylene glycol and its ethers, such as propylene glycol monomethyl ether, butylene glycol, dipropylene glycol, diethylene glycol alkyl ethers, such as diethylene glycol monoethyl ether and monobutyl ether, hydrocarbons such as straight chain hydrocarbons, mineral oil, polybutene, hydrogenated polyisobutene, hydrogenated polydecene, polydecene, squalane, petrolatum, isoparaffins, and mixtures, thereof.

The at least one solvent may, for example, be present in an amount ranging from 0.5% to 70% by weight, such as from 2% to 60% by weight, preferably from 5 to 50% by weight, relative to the total weight of the developer composition.

The developer composition may be in the form of a powder, gel, liquid, foam, lotion, cream, mousse, and emulsion.

The pH of the developer composition can range from 2 to 12, such as from 6 to 11, and it may be adjusted to the desired value using basifying/alkalizing agents that are well known in the art of dyeing hair.

Direct Dyes

A direct dye is a colored substance that does not require the use of an oxidizing agent in order to reveal its color. Suitable direct dyes which may be used according to the present invention may be chosen from acidic (anionic), basic (cationic), and neutral dyes.

“Acidic dye” is generally intended to mean a dye containing at least one COOH, SO₃H, PO₃H, or PO₄H₂ group, it being possible for said groups to exist in the form of salts. “Salts” is generally intended to mean salts of metals (for example, alkali metals or alkaline earth metals), salts of an organic amine that is optionally hydroxylated. Such dyes are also referred to as anionic dyes.

The acidic dyes that can be used in the context of this invention can be chosen from acidic nitro dyes, acidic azo dyes, acidic azine dyes, acidic triarylmethane dyes, acidic quinone dyes, acidic indo-amine dyes and acidic natural dyes, and mixtures thereof.

“Basic dyes” is generally intended to mean a dye that has at least one group bearing a positive charge, such as an ammonium group or a quaternized nitrogen atom in a ring. Such dyes are also referred to as cationic dyes.

The basic dyes that can be used in the context of this invention can be chosen from nitrobenzene dyes, azo dyes, azomethine dyes, methine dyes, tetraazapentamethine dyes, anthraquinone dyes, naphthoquinone dyes, benzoquinone dyes, phenothiazine dyes, indigoid dyes, xanthene dyes, phenanthridine dyes, phthalocyanin dyes, triarylamethane-derived dyes and basic natural dyes, and mixtures thereof.

Preferably, the direct dyes may be present in amounts ranging from 0.001% to 30% by weight, preferably from 0.01% to 20% by weight, more preferably from 0.1% to 10% by weight, based on the total weight of the coloring composition.

Pigments

The pigments, which may be used according to the present disclosure, may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, silica, ferric blue, and mixtures thereof. Representative examples of organic pigments include carbon black, pigments of D & C type, and lakes based on cochineal carmine, barium, strontium, calcium, and aluminum. Other examples of pigments are ultramarines, HC Blue No. 14, Ext. Yellow 7, Yellow 10 Lake, and acid violet 43.

If present, the pigments may be present in the composition in a concentration ranging up to 50% by weight of the total weight of the coloring composition, such as from 0.5% to 40%, and further such as from 2% to 30%.

Natural Colorants

Representative natural colorants include those disclosed in US patent application publication no. US20030159221, the entire contents of which is hereby incorporated by reference. For the purposes of the invention, the expression “natural colorant” means compounds that exist in nature, whether they have been obtained by extraction or reproduced chemically. Examples of natural direct dyes that may be used according to the invention include lawsone, juglone, alizarin, purpurin, carminic acid, kermesic acid, purpurogallin, protocatechaldehyde, indigo, isatin, curcumin, spinulosin and apigenidin. It is also possible to use extracts or decoctions containing these natural dyes and especially henna-based poultices or extracts.

If present, the natural colorants may be present in the composition in a concentration ranging up to 50% by weight of the total weight of the coloring composition, such as from 0.05% to 40%, and further such as from 2% to 30%.

Liposoluble Dyes

Representative liposoluble dyes which may be used according to the present invention include Sudan Red, DC Red 17, DC Green 6, β-carotene, soybean oil, Sudan Brown, DC Yellow 11, DC Violet 2, DC Orange 5, DC Blue No. 14, annatto, and quinoline yellow. The liposoluble dyes, when present, may have a concentration ranging up to 20% by weight of the total weight of the coloring composition, such as from 0.0001% to 6% by weight.

Nacreous Pigments

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, may be present in the color base composition in a concentration ranging up to 50% by weight of the total weight of the coloring composition, such as from 0.1% to 20%, preferably from 0.1% to 15%.

Leuco Dyes

Representative leuco dyes include those disclosed in US patent application publication no. 20040194231, the entire contents of which is hereby incorporated by reference. Leuco dyes are usually only slightly colored or are not colored at all and can be converted by simple oxidation in air or in the presence of an oxidizing agent into a triheteroylmethane compound. Examples of leuco dyes and corresponding triheteroylmethane compounds include 1H-Benzo[ij]quinolizinium, 9-[bis(2,3,6,7-tetrahydro-1H,5H-benzo[ij-]quinolizin-9-yl)methylene]-2,3,5,6,7,9-hexahydro-chloride; 5H-Benzo[a]carbazolium, 11-ethyl-5-[(11-ethyl-11H-benzo[a]carbazol-5-yl)(1-ethyl-1,2,3,4-tetrahydro-5-quinolinyl)methylene]-; Pyrrolo[3,2,1-ij]quinolinium, 8-[bis(1,2,5,6-tetrahydro-4H-pyrrolo[3,2,1-ij]quinolin-8-yl)methylene]-1,2,4,5,6,8-hexahydro-; Tri(9-ethyl-9H-carbazol-3-yl)methane; bis(6-Chloro-9-ethyl-9H-carbazol-3-yl)-(9-ethyl-9H-carbazol-3-yl)methane; bis(1-(4-sulfo-butyl)-2,3,4,6-tetrahydro-quinolinium)-pyrid-4-yl-methane; bis(1-ethyl-2-methyl-1H-indol-3-yl)-(9-ethyl-9H-carbazol-3-yl)methane; Tri(7-ethyl-7H-benzo[c]carbazol-10-yl)methane; bis((6-dimethylamino-3-methyl-1H-indol-2-yl)-2-furylmethane; bis((6-dimethylamino-3-methyl-1H-indol-2-yl)-(pyrid-4-yl)methane; bis(1-ethyl-2-methyl-1H-indol-3-yl)-2-thienylmethane; 3-[(1-ethyl-2-methyl-1H-indol-3-yl)-(9-ethyl-9H-carbazol-3-yl)methylene]-1-ethyl-2-methyl-3H-indolium; and 3-[(1-ethyl-2-methyl-1H-indol-3-yl)-2-thienyl)methylene]-1-ethyl-2-methyl-3H-indolium.

Optical Lightening Colorants

Representative optical lightening colorants include those disclosed in US patent application publication no. US20040205905, the entire contents of which is hereby incorporated by reference.

### Cosmetically Acceptable Carriers

The cosmetically acceptable carrier can be water and/or an organic solvent. Suitable organic solvents include alcohols, such as ethanol, isopropyl alcohol, benzyl alcohol and phenyl ethyl alcohol; glycols and glycol ethers, such as propylene glycol, hexylene glycol, ethylene glycol monomethyl, monoethyl or monobutyl ether, propylene glycol and its ethers, such as propylene glycol monomethyl ether, butylene glycol, dipropylene glycol, and also diethylene glycol alkyl ethers, such as diethylene glycol monoethyl ether and monobutyl ether; hydrocarbons such as straight chain hydrocarbons, mineral oil, polybutene, hydrogenated polyisobutene, hydrogenated polydecene, polydecene, squalene, petrolatum isoparaffins, esters, silicone oils, waxes, and mixtures, thereof.

Moreover, the cosmetic composition can be water and/or other cosmetically acceptable solvents such as lower alcohols, polyols such as glycerol, glycols such as butylene glycol, isoprene glycol, propylene glycol, or polyethylene glycols such as PEG-8, sorbitol, sugars such as glucose, fructose, maltose, lactose or sucrose, and mixtures thereof. Other cosmetically acceptable carriers may include mineral oils, paraffin, silicone oils, synthetic or natural esters, and waxes.

The cosmetically acceptable carrier is typically present in an amount of from about 0.1% to about 90%, from about 0.1% to about 50%, from about 0.1 to about 25%, or from about 0.1% to about 15% by weight of the cosmetic composition. In one embodiment, the amount of cosmetically acceptable carrier is equal to or less than the combined amount of silicone amine and anionic silicone.

The cosmetic composition can comprise a cosmetically acceptable carrier in the amount of about 95%, 90%, 80%, 70%, 60%, 50%, 40%, 30%, 20%, 10%, 5% or less based on the total weight of the cosmetic composition. The cosmetic composition can comprise a cosmetically acceptable carrier in the amount of about 5%, 10%, 20%, 30%, 40%, 50%, 60%, 70%, 80%, 90%, 95%, or more based on the total weight of the cosmetic composition. Additionally the amount of the cosmetically acceptable carrier can be any combination of the above values, for example from about 20% to about 95%, or from about 50% to about 60% by weight based on the total weight of the cosmetic compositions.

In one embodiment, the cosmetically acceptable carrier comprises water and at least one co-solvent chosen from an organic solvent. The at least one co-solvent may be present in an amount ranging from about 1% to about 50% by weight, from about 1% to about 30% by weight, such as from about 2% to about 20% by weight, relative to the total weight of the cosmetic composition.

### Conditioning Agents

Conditioning agents may also be employed in order to impart added conditioning benefits to the hair coloring composition. The conditioning agents useful in the present hair coloring compositions include cationic surfactants, silicone compounds (which are different than the (a) a silicone amine and (b) an anionic silicone), polyalkylene glycols and mixtures thereof. Moreover, the condition agents can be mono long-chain ammonium compounds, hydrophilically substituted cationic surfactants, hydrophilically substituted silicone compounds, polyalkylene glycols, and mixtures thereof.

The type of conditioning agent to be included in the hair coloring composition depends on the desired characteristics of the product. Water soluble conditioning agents are often used. A combination of conditioning agents can also be used to provide benefits provided by the different conditioning agents. Furthermore, conditioning agents which are less water soluble can be used in combination with highly water soluble conditioning agents.

Cationic surfactants may be used as conditioning agents. Suitable cationic surfactants include, but are not limited to, those generally described as mono long-chain ammonium compounds. Nonlimiting examples of such cationic surfactants include: cetyl trimethyl ammonium chloride available, for example, with tradename CA-2350 from Nikko Chemicals and CTAC 30KC available from KCl, stearyl trimethyl ammonium chloride with tradename Arquad 18/50 available from Akzo Nobel, hydrogenated tallow alkyl trimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, and N-(stearoyl colamino formyl methy) pyridinium chloride.

Also useful are hydrophilically substituted cationic surfactants in which at least one of the substituents contain one or more aromatic, ether, ester, amido, or amino moieties present as substituents or as linkages in the radical chain. Nonlimiting examples of hydrophilically substituted cationic surfactants useful in the present disclosure include the materials having the following CTFA designations: quaternium-16, quaternium-26, quaternium-27, quaternium-30, quaternium-33, quaternium-43, quaternium-52, quaternium-53, quaternium-56, quaternium-60, quaternium-61, quaternium-62, quaternium-70, quaternium-71, quaternium-72, quaternium-75, quaternium-76 hydrolyzed collagen, quaternium-77, quaternium-78, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, and quaternium-79 hydrolyzed wheat protein, quaternium-80, quaternium-81, quaternium-82, quaternium-83, quaternium-84, and mixtures thereof.

In one embodiment, the hydrophilically substituted cationic surfactants include dialkylamido ethyl hydroxyethylmonium salt, dialkylamidoethyl dimonium salt, dialkyloyl ethyl hydroxyethylmonium salt, dialkyloyl ethyldimonium salt, alkyl amidopropyl trimonium salt, polyoxyethylene alkyl ammonium salt, and mixtures thereof; for example, commercially available under the following tradenames; VARISOFT 110, VARISOFT PATC, VARIQUAT K1215 and 638 from Witco Chemical, ETHOQUAD 18/25, ETHOQUAD O/12PG, ETHOQUAD C/25, and ETHOQUAD S/25 from Akzo, DEHYQUART SP from Cognis, and MONAQUAT ISEIS, and MONAQUAT SL-5 available from Uniqema.

The polyalkylene glycols useful as conditioning agents include those which are soluble or dispersible in water, such as polyethylene glycols.

Polyalkylene glycols having a molecular weight of more than about 100 are also useful. Ethylene oxide polymers generally have good water solubility, dispersibility, and transparency. Polyethylene-polypropylene glycols and polyoxyethylene-polyoxypropylene copolymer polymers having good dispersibility and transparency can also be useful.

Suitable silicone compounds, which are different than the (a) a silicone amine and (b) an anionic silicone include, but are not limited to, polyalkyl siloxanes, polyaryl siloxanes, polyalkylaryl siloxanes, polyether siloxane copolymers, and mixtures thereof. Other insoluble, nonvolatile silicone fluids having conditioning properties can also be used.

Silicone compounds useful in the hair coloring compositions include, for example, polyalkyl or polyaryl siloxanes represented by the formula below.

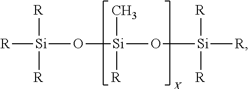

R is aliphatic, alkyl or alkenyl, or aryl, R can be substituted or unsubstituted, and x is an integer from 1 to about 8,000. Suitable unsubstituted R groups include alkoxy, aryloxy, alkaryl, arylalkyl, arylalkenyl, alkamino, and ether-substituted, hydroxyl-substituted, and halogen-substituted aliphatic and aryl groups.

The two R groups on the silicone atom of each monomeric silicone unit may represent the same group or different groups. The alkyl and alkenyl substituents can be C₁-C₅ alkyls and alkenyls, C₁-C₄ alkyls and alkenyls, or C₁-C₂ alkyls and alkenyls. The aliphatic portions of other alkyl alkenyl or alkynyl-containing groups (such as alkoxy, alkaryl, and alkamino) can be straight or branched chains and can have from one to five carbon atoms, from one to four carbon atoms, or from one to three carbon atoms, and even from one to two carbon atoms. The R substituents can also be substituted with other groups, such as halogens (e.g. chloride, fluoride, and bromide), halogenated aliphatic or aryl groups, and hydroxy (e.g. hydroxy substituted aliphatic groups). Suitable halogenated R groups can include, for example, tri-halogenated (e.g., fluoro) alkyl groups such as —R¹—C(F)₃, wherein R¹ is C₁-C₃ alkyl. Examples of such polysiloxanes include polymethyl-3,3,3 trifluoropropylsiloxane.

Suitable R groups include methyl, ethyl, propyl, phenyl, methylphenyl and phenylmethyl. Other suitable R groups include methyl, methoxy, ethoxy, propoxy, and aryloxy. Non-limiting examples of silicone oils include polydimethyl siloxane, polydiethyl siloxane, polydipropyl siloxane, polymethylethyl siloxane, polymethylpropylsiloxane, and polymethylphenylsiloxane.

The silicone compound (which are different than the (a) a silicone amine and (b) an anionic silicone) can be, for example, a polyalkyl siloxane, a polyaryl siloxane, a polyalkylaryl siloxane, a polyether siloxane copolymer, and mixtures thereof. In some aspects, the silicone oil can be, for example, a polydimethyl siloxane, a polydiethyl siloxane, a polydipropyl siloxane, a polymethylethyl siloxane, a polymethylpropylsiloxane, a polymethylphenylsiloxane, and mixtures thereof. In other aspects, the silicone compound (which are different than the (a) a silicone amine and (b) an anionic silicone) is polydimethyl siloxane.

The hair coloring compositions typically comprise from about 1% to about 90%, from about 1% to about 75%, from about 1% to about 50%, or from about 1% to about 25% by weight of a conditioning agent based on the total weight of the hair coloring composition. The hair coloring composition can comprise condition agent in the amount of about 95%, 90%, 80%, 70%, 60%, 50%, 40%, 30%, 20%, 10%, 5% or less based on the total weight of the hair coloring composition. The hair coloring composition can comprise conditioning agent in the amount of about 5%, 10%, 20%, 30%, 40%, 50%, 60%, 70%, 80%, 90%, 95%, or more based on the total weight of the hair coloring composition. Additionally the amount of conditioning agent can be any combination of the above values, for example from about 20% to about 95%, or from about 50% to about 60% by weight based on the total weight of the hair coloring compositions.

### Auxiliary Ingredients

The compositions of the present disclosure can further comprise at least one auxiliary ingredient including film forming agents, surfactants, conditioning agents, skin active agents and mixtures thereof.

The composition of the present disclosure may contain at least one film forming agent. Film-forming agents useful herein are neutralized, non-neutralized or partially neutralized, polymers and resins, wherein the polymers and resins include but are not limited to those containing carboxyl moieties, such as acrylates and other carboxy polymers. Examples of suitable water soluble film forming agents include, for example, PVP, PVP/VA, acrylates, polyesters, polyurethranes, polyimides, polysulfonates, guars, starches and the like. Typically, water-insoluble polymers and resins have to be neutralized to about 90% of their carboxyl moieties to make them water soluble for the purpose of formulating products in aqueous solution and for the purpose of making products which have good non-build-up properties, i.e., can be easily washed off the hair after use.

The following are examples of film forming agents that can be employed by the present disclosure. The list is not intended to be limiting: AMPHOMER® LV-71 from National Starch (octylacrylamide/acrylates/butylaminoethyl methacrylate copolymer), OMNIREZ®-2000 from ISP (PVM/MA half ethyl ester copolymer), RESYN® 28-2930 from National Starch (Vinyl acetate/crotonates/vinyl neodecanoate copolymer), LUVIMER® 100P from BASF (t-butyl acrylate/ethyl acrylate/methacrylic acid), ULTRAHOLD® STRONG from BASF (acrylic acid/ethyl acrylate/t-butyl acrylamide), SALCARE® SC60 from Ciba (Acrylamidopropyltrimonium Chloride/Acrylamide Copolymer), BALANCE® CR from National Starch (Acrylates Copolymer), AMPHOMER® 28-4961 from National Starch (Acrylates/Octylacrylamide Copolymer), TORAY SETS®IL 301 from Dow Corning (Acrylates/Octylacrylamide/Diphenyl Amodimethicone Copolymer), DIAFORMER® Z-632N from Clariant (Acrylates/Stearyl Acrylate/Ethylamine Oxide Methacrylate Copolymer), ULTRAHOLD® 8 from BASF (Acrylates/t-Butylacrylamide Copolymer), MEXOMERE® PQ from Chimex (Allyl Stearate/VA Copolymer), FIXATE® G-100 from Noveon® (AMP-Acrylates/Allyl Methacrylate Copolymer), GANTREZ® A-425 from ISP (Butyl Ester of PVM/MA Copolymer), GANEX®P-904 from ISP (Butylated PVP), AMAZE® from National Starch (Corn Starch Modofied), MEXOMERE® PL from Chimex (Diethylene Glycolamine/Epichlorohydrin/piperazine Copolymer), EASTMAN® AQ POLYMER from Eastman (Diglycol/CHDM/Isophthalate/SIP Copolymer), JAGUAR® C 13S from Rhodia (Guar Hydroxylpropyl Trimonium Chloride), AQUAFLEX® FX-64 from ISP (Isobutylene/Ethylmaleimide/Hydroxyethylmaleimide Copolymer), LUVIFLEX® SILK from BASF (PEG/PPG-25/25 Dimethicone/Acrylates Copolymer), AQUAFLEX® XL-30 from ISP (Polyimide-1), LUVISET® P.U.R from BASF (Polyurethrane-1), LUVISKOL® PLUS from BASF (Polyvinylcaprolactam), AQUAFLEX® SF-40 from ISP (PVP/Vinylcaprolactam/DMAPA Acrylates Copolymers), ADVANTAGE® PLUS from ISP (VA/Butyl Maleate/Isobornyl Acrylate Copolymer), MEXOMERE® PW from Chimex (VA/Vinyl Butyl Benzoate/Crotonates Copolymer), GAFFIX VC-713 from ISP (Vinyl Caprolactam/VP/Dimethylaminoethyl Methacrylate Copolymer), COPOLYMER 845 from ISP (VP/Dimethylaminoethylmethacrylate Copolymer), GANEX® V-516 from ISP (VP/Hexadecene Copolymer), LUVISKOL® VA 64 from BASF (VP/VA Copolymer).

Unneutralized or partially neutralized water-insoluble latexes can also be used as invention film forming agents. Included are the following latexes: AMERHOLD® DR-25 from Amerchol (acrylic acid/methacrylic acid/acrylates/methacrylates), LUVIMER® 36D from BASF (ethyl acrylate/t-butyl acrylate/methacrylic acid), and ACUDYN E® 258 from Rohm & Haas (acrylic acid/methacrylic acid/acrylates/methacrylates/hydroxy ester acrylates).

In one embodiment, the film forming agent is selected from the group consisting of polysilicone 8, polydimethylsiloxane, dimethylsiloxane/3-thiopropyl methyl siloxane copolymer, vinylpyyrolidone/vinylacetate copolymer, polyvinyacetate, starch, polyquaternium-4, polyquaternium-11, acrylates/steareth-2 methacrylate crosspolymer, vinylacetate/vinyl neodecanoate copolymer, polyester-5, cetyl ethylhexanoate, vinyl acetate, crotonate/vinyl neodecanoate copolymer, 2-acryamido-2-methyl propane sulfonic acid (AMPS)/acrylic acid (AA) copolymer, AMPS/AA/acryl methacrylate copolymer, polyacrylamide, C₁₃-C₁₄ isoparaffin, laureth-7, octylacrylamide, and acrylate/butylaminoethylmethacrylate copolymer.

The film forming agent may be employed in an amount sufficient to impart and/or maintain a shape on the hair. Typically, it will be employed in an amount of from greater than 0 to 30% by weight, preferably from 1 to 10% by weight, and more preferably from 1 to 5% by weight, based on total weight of composition.

The surfactants of the present disclosure may be chosen from anionic surfactants, cationic surfactants, nonionic surfactants, amphoteric surfactants, zwitterionic surfactants and mixtures thereof.

In general, non-limiting examples of surfactants include an amphoteric surfactant chosen from allyl betaines, alkylamphoglycinates, alkylamphopropylsulfonates, alkylamphopropionates, alkylamphocarboxyglycinates, alkyl sultaines, hydroxysultaines, alkylamidobetaines, betaines, alkyl propionates, cocamidoalkyl betaines, cocamidoalkyl hydroxysultaines, cocamidoalkyl dimethylamine propionates, alkylamido alkylamines, their derivatives and mixtures thereof.

Suitable amphoteric surfactants include an amphoteric surfactant chosen from cocamidoethyl betaine, cocamidopropyl betaine, cocamidopropyl hydroxysultaine, cocamidopropyl dimethylamine propionate, cocoamphoglycinate, cocoamphocarboxypropionate, cocoamphocarboxyglycinate, coco-betaine, cocoamphopropionate, cocoamphopropylsulfonate and mixtures thereof.

Examples of amphoteric surfactants include but are not limited to lauryl betaine, lauroamphoglycinate, lauroamphopropylsulfonate, lauroamphopropionate, lauroampho-carboxyglycinate, lauryl sultane, myristamidopropyl betaine, myristyl betaine, myristoamphoglycinate, myristyl propionate, stearoamphoglycinate, stearoamphopropionate, stearoamphopropylsulfonate, stearyl betaine, cocamidoethyl betaine, cocamidopropyl betaine, cocamidopropyl hydroxysultaine, cocamidopropyl dimethylamine propionate, cocoamphoglycinate, cocoamphocarboxypropionate, cocoamphocarboxyglycinate, coco-betaine, cocoamphopropionate, cocoamphopropylsulfonate and mixtures thereof.

In one embodiment, the at least one surfactant includes an amphoteric surfactant, cocamidopropyl hydroxysultaine.

Non-limiting examples of surfactants include alkyl sulfates, alkyl ether sulfates, ether sulfates, alkylamido ether sulfates, alkylaryl polyether sulfates, monoglyceride sulphates, alkyl sulfonates, alkyl phosphates, alkylamide sulfonates, alkylaryl sulfonates, α-olefin sulfonates, paraffin sulfonates, alkyl sulfosuccinates, alkyl ether sulfosuccinates, alkylamide sulfosuccinates, alkyl sulfosuccinamates, alkyl sulfoacetates, alkyl ether phosphates, acyl sarcosinates, acyl isethionates, acylglutamates, N-acyltaurates, acyllatylates, fatty acids, alkyl-D-galactosiduronic acids, polyoxyalkylenated carboxylic alkyl ether acids, polyoxyalkylenated carboxylic alkylaryl ether acids, C6-24 alkyl monoesters of polyglycoside dicarboxylic acids, polyoxyalkylenated (C6-24 alkyl)(C6-24 aryl)ether carboxylic acids, polyoxyalkylenated (C6-24 alkyl) amido ether carboxylic acids alkyl glucoside citrates, alkyl polyglycoside tartrates, alkyl polyglycoside sulfosuccinates, alkyl sulfosuccinamates, their derivatives, their salts and mixtures thereof.

Examples of suitable anionic surfactant include sodium lauryl ether sulfate, sodium lauryl sulfate, ammonium lauryl ether sulfate, ammonium lauryl sulfate, disodium laureth sulfosuccinate, and mixtures thereof.

Conditioning agents may also be employed in the composition of the present disclosure in order to impart added conditioning benefits to the keratinous substrates. The conditioning agents useful in the present compositions include cationic surfactants, silicone compounds (which are different than the (a) a silicone amine and (b) an anionic silicone), polyalkylene glycols and mixtures thereof. Moreover, the conditioning agents can be mono long-chain ammonium compounds, hydrophilically substituted cationic surfactants, hydrophilically substituted silicone compounds, polyalkylene glycols, and mixtures thereof.

“Conditioning” as used herein means imparting to at least one keratinous fiber at least one property chosen from combability, manageability, moisture-retentivity, luster, shine, softness, suppleness and silkiness. The type of conditioning agent to be included in the composition depends on the desired characteristics of the product. Water soluble conditioning agents are often used. A combination of conditioning agents can also be used to provide benefits provided by the different conditioning agents. Furthermore, conditioning agents which are less water soluble can be used in combination with highly water soluble conditioning agents.

Cationic surfactants may be used as conditioning agents. Suitable cationic surfactants include, but are not limited to, those generally described as mono long-chain ammonium compounds. Nonlimiting examples of such cationic surfactants include: cetyl trimethyl ammonium chloride available, for example, with tradename CA-2350 from Nikko Chemicals and CTAC 30KC available from KCl, stearyl trimethyl ammonium chloride with tradename Arquad 18/50 available from Akzo Nobel, hydrogenated tallow alkyl trimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, and N-(stearoyl colamino formyl methy) pyridinium chloride.

Also useful are hydrophilically substituted cationic surfactants in which at least one of the substituents contain one or more aromatic, ether, ester, amido, or amino moieties present as substituents or as linkages in the radical chain. Nonlimiting examples of hydrophilically substituted cationic surfactants useful in the present disclosure include the materials having the following CTFA designations: quaternium-16, quaternium-26, quaternium-27, quaternium-30, quaternium-33, quaternium-43, quaternium-52, quaternium-53, quaternium-56, quaternium-60, quaternium-61, quaternium-62, quaternium-70, quaternium-71, quaternium-72, quaternium-75, quaternium-76 hydrolyzed collagen, quaternium-77, quaternium-78, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, and quaternium-79 hydrolyzed wheat protein, quaternium-80, quaternium-81, quaternium-82, quaternium-83, quaternium-84, and mixtures thereof.

In one embodiment, the hydrophilically substituted cationic surfactants include dialkylamido ethyl hydroxyethylmonium salt, dialkylamidoethyl dimonium salt, dialkyloyl ethyl hydroxyethylmonium salt, dialkyloyl ethyldimonium salt, alkyl amidopropyl trimonium salt, polyoxyethylene alkyl ammonium salt, and mixtures thereof; for example, commercially available under the following tradenames; VARISOFT 110, VARISOFT PATC, VARIQUAT K1215 and 638 from Witco Chemical, ETHOQUAD 18/25, ETHOQUAD O/12PG, ETHOQUAD C/25, and ETHOQUAD S/25 from Akzo, DEHYQUART SP from Cognis, and MONAQUAT ISEIS, and MONAQUAT SL-5 available from Uniqema.

The polyalkylene glycols useful as conditioning agents include those which are soluble or dispersible in water, such as polyethylene glycols.

Polyalkylene glycols having a molecular weight of more than about 100 are also useful. Ethylene oxide polymers generally have good water solubility, dispersibility, and transparency. Polyethylene-polypropylene glycols and polyoxyethylene-polyoxypropylene copolymer polymers having good dispersibility and transparency can also be useful.

Suitable silicone compounds, which are different than the (a) a silicone amine and (b) an anionic silicone include, but are not limited to, polyalkyl siloxanes, polyaryl siloxanes, polyalkylaryl siloxanes, polyether siloxane copolymers, and mixtures thereof. Other insoluble, nonvolatile silicone fluids having conditioning properties can also be used.

Silicone compounds useful in the compositions include, for example, polyalkyl or polyaryl siloxanes represented by the formula below.

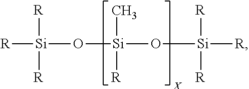

R is aliphatic, preferably alkyl or alkenyl, or aryl, R can be substituted or unsubstituted, and x is an integer from 1 to about 8,000. Suitable unsubstituted R groups include alkoxy, aryloxy, alkaryl, arylalkyl, arylalkenyl, alkamino, and ether-substituted, hydroxyl-substituted, and halogen-substituted aliphatic and aryl groups.

The two R groups on the silicone atom of each monomeric silicone unit may represent the same group or different groups. The alkyl and alkenyl substituents can be C₁-C₅ alkyls and alkenyls, C₁-C₄ alkyls and alkenyls, or C₁-C₂ alkyls and alkenyls. The aliphatic portions of other alkyl alkenyl or alkynyl-containing groups (such as alkoxy, alkaryl, and alkamino) can be straight or branched chains and can have from one to five carbon atoms, from one to four carbon atoms, or from one to three carbon atoms, and even from one to two carbon atoms. The R substituents can also be substituted with other groups, such as halogens (e.g. chloride, fluoride, and bromide), halogenated aliphatic or aryl groups, and hydroxy (e.g. hydroxy substituted aliphatic groups). Suitable halogenated R groups can include, for example, tri-halogenated (e.g., fluoro) alkyl groups such as —R¹—C(F)₃, wherein R¹ is C₁-C₃ alkyl. Examples of such polysiloxanes include polymethyl-3,3,3 trifluoropropylsiloxane.

Suitable R groups include methyl, ethyl, propyl, phenyl, methylphenyl and phenylmethyl. Other suitable R groups include methyl, methoxy, ethoxy, propoxy, and aryloxy. Non-limiting examples of silicone oils include polydimethyl siloxane, polydiethyl siloxane, polydipropyl siloxane, polymethylethyl siloxane, polymethylpropylsiloxane, and polymethylphenylsiloxane.

The silicone compound (which are different than the (a) a silicone amine and (b) an anionic silicone) can be, for example, a polyalkyl siloxane, a polyaryl siloxane, a polyalkylaryl siloxane, a polyether siloxane copolymer, and mixtures thereof. In some aspects, the silicone oil can be, for example, a polydimethyl siloxane, a polydiethyl siloxane, a polydipropyl siloxane, a polymethylethyl siloxane, a polymethylpropylsiloxane, a polymethylphenylsiloxane, and mixtures thereof. In other aspects, the silicone compound (which are different than the (a) a silicone amine and (b) an anionic silicone) is polydimethyl siloxane.

The conditioning agent may also be chosen from amino acids, proteins, extracts, fats, oils, esters, hydrocarbons, quats, polyquats, polyols, humectants, alkanolamides, fatty acids, ketones and mixtures thereof.

The compositions typically comprise from about 1% to about 90%, from about 1% to about 75%, from about 1% to about 50%, or from about 1% to about 25% by weight of a conditioning agent based on the total weight of the composition.

The composition can comprise the conditioning agent in the amount of about 95%, 90%, 80%, 70%, 60%, 50%, 40%, 30%, 20%, 10%, 5% or less based on the total weight of the composition. The composition can comprise the conditioning agent in the amount of about 5%, 10%, 20%, 30%, 40%, 50%, 60%, 70%, 80%, 90%, 95%, or more based on the total weight of the composition. Additionally the amount of conditioning agent can be any combination of the above values, for example from about 20% to about 95%, or from about 50% to about 60% by weight based on the total weight of the compositions.

The composition of the present disclosure contains at least one skin active agent. The at least one skin active agent includes photoprotective agents, self-tanning agents, desquamating agents, depigmenting agents, moisturizing agents, skin lightening agents, anti-aging ingredients, anti-wrinkle agents, anti-dandruff agents and mixtures thereof. Non-limiting examples of the at least one skin active agent include α-hydroxy acids, β-hydroxy acids, α-keto acids, β-keto acids, retinoids, anthralins, anthranoids, peroxides, lithium salts, antimetabolites, vitamin D, antioxidants, ingredients that could promote moisturization and desquamating agents.

As active agents that may be used in the composition of the present disclosure, examples that may be mentioned include moisturizers, for example, protein hydrolysates and polyols such as glycerol, glycols, for instance polyethylene glycols, and sugar derivatives; natural extracts; anti-inflammatory agents; procyannidol oligomers; vitamins, for instance vitamin A (retinol), vitamin C (ascorbic acid), vitamin E (tocopherol), vitamin B5 (panthenol), vitamin B3 (niacinamide), derivatives of these vitamins (especially esters) and mixtures thereof; urea; caffeine; depigmenting agents such as kojic acid, hydroquinone and caffeic acid; salicylic acid and its derivatives; α-hydroxy acids such as lactic acid and glycolic acid and derivatives thereof; retinoids such as carotenoids and vitamin A derivatives; sunscreens; self-tanning agents; hydrocortisone; melatonin; algal, fungal, plant, yeast or bacterial extracts; enzymes; DHEA and its derivatives and metabolites; antibacterial active agents, for instance 2,4,4′-trichloro-2′-hydroxydi-phenyl ether (or Triclosan), 3,4,4′-trichloro-carbanilide (or Triclocarban) and the acids indicated above and especially salicylic acid and its derivatives; mattifying agents and mixtures thereof.

Preferred embodiments of the compositions of the present disclosure include skin active agents chosen from hydroxy acids, vitamins, sunscreens, UV filters, humectants, glycols, polyols, self-tanning ingredients, antioxidants and mixtures thereof.

Other preferred embodiments of the compositions of the present disclosure include skin active agents chosen from photoprotective agents, self-tanning agents, desquamating agents, depigmenting agents, moisturizing agents, skin lightening agents, anti-aging ingredients, anti-wrinkle agents, anti-dandruff agents and mixtures thereof.

The compositions typically comprise from about 0.01% to about 90%, from about 0.1% to about 75%, from about 0.5% to about 50%, or from about 1% to about 25% by weight of the at least one skin active agent based on the total weight of the composition.

The cosmetic compositions typically comprise from about 0.01% to about 90%, from about 0.05% to about 75%, from about 0.1% to about 50%, or from about 0.5% to about 25% by weight of the at least one auxiliary ingredient based on the total weight of the composition.

### Additional Components

It is possible to incorporate into the compositions described above additional components such as shine agents, thickeners and/or oils, and other components. Other additional components include shine agents, thickeners and/or oils, viscosity modifiers, rheology-modifying agents, plant extracts, humectants, reducing agents, sequestering agents, softeners, antifoams, emollients, basifying and acidifying agents, gelling agents, wetting agents, spreading agents, dispersants, plasticizers, preservatives, direct dyes or oxidation dyes, colorants, pigments, mineral fillers, clays, colloidal minerals, nacres, nacreous agents, emulsifying agents, fragrances, peptizers, preserving agents, fixing or non-fixing polymers, ceramides, proteins, aliphatic or aromatic alcohols, volatile silicones, hydrocarbons, mineral, organic or plant oils, waxes, paraffins, fatty acids, associative or non-associative thickening polymers, fatty amides, fatty esters, and fatty alcohols.

The composition of the present disclosure may contain at least one shine agent chosen from silicones, alkoxylated silicones, oils, ethoxylated oils, fats, esters, transesters, hydrocarbons, quats and mixtures thereof. Suitable silicone compounds, which are different than the (a) a silicone amine and (b) an anionic silicone include, but are not limited to, Dimethicone, Dimethiconol, Cyclomethicone, Phenyltrimethicone, Aminopropyl Phenyltrimethicone, Trimethyl Pentaphenyl Trisiloxane, Cetyl Dimethicone, Alkyl Dimethicone, and mixtures thereof.

Other shine agents of the present disclosure may include olive oil, Jojoba oil, Apricot oil, Avocado oil, Castor oil, Lanolin, Squalane, Capric/Caprylic Triglyceride, Octyl Palmitate, Isopropyl Palmitate, Isopropyl Myristate, Mineral oil, Petrolatum, Polyquaternium-4, Polyquaternium-11, Behentrimonium Methosulfate, Benetrimonium Chloride and mixtures thereof.

The shine agent may be employed in an amount from about 0.01% to about 95% by weight based on the total weight of the composition. Typically, the shine agent is added in an amount from about 1% to about 50% by weight based on the total weight of the composition and more typically from about 5% to about 20% by weight based on the total weight of the composition.

Examples of thickeners include mineral salts such as sodium chloride; oxyethylenated molecules and especially ethoxylated alkyl or acyl derivatives of polyols which may be, in particular, oxyethylenated derivatives of fatty acid esters or of fatty alcohol ethers and of a polyol such as glycerol, sorbitol, glucose or pentaerythritol. Examples of compounds of this type that may be mentioned include oxyethylenated (200 EO) glyceryl stearate, such as the product sold under the name Simulsol® 220 by the company SEPPIC, oxyethylenated (150 EO) pentaerythrityl tetrastearate, such as the product sold under the name Crothix® by the company Croda, oxyethylenated (120 EO) methylglucose dioleate, such as the product sold under the name Glucamate DOE-120 Vegetal® by the company Amerchol, or oxyethylenated (160 EO) sorbitan triisostearate, such as the product sold under the name Rheodol® TW 15399C by the company Kao Chemicals.

Thickeners are typically present in concentrations ranging, for example, from about 0.05% to about 10% by weight, from about 0.2% to about 5% by weight, from about 0.2% to about 2% by weight based on the total weight of the composition.

Oils useful in the compositions include hydrocarbon-based oils such as liquid triglycerides of fatty acids of 4 to 10 carbon atoms, such as heptanoic or octanoic acid triglycerides or alternatively, for example, sunflower oil, corn oil, soybean oil, marrow oil, grapeseed oil, sesame oil, hazelnut oil, apricot oil, macadamia oil, arara oil, castor oil, avocado oil, caprylic/capric acid triglycerides such as those sold by the company Stearineries Dubois or those sold under the names Miglyol 810, 812 and 818 by the company Dynamit Nobel, jojoba oil or karite butter; synthetic esters and ethers in particular of fatty acids, such as the oils of formulae R₁COOR.₂ and R₁OR₂ in which R₁ represents a fatty acid residue containing from 8 to 29 carbon atoms and R₂ represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate or isostearyl isostearate; hydroxylated esters such as isostearyl lactate, octyl hydroxystearate, octyldodecyl hydroxystearate, diisostearyl malate, triisocetyl citrate, and fatty alcohol heptanoates, octanoates and decanoates; polyol esters such as propylene glycol dioctanoate, neopentyl glycol diheptanoate and diethylene glycol diisononanoate; and pentaerythritol esters such as pentaerythrityl tetraisostearate; linear or branched hydrocarbons of mineral or synthetic origin, such as volatile or non-volatile liquid paraffins and derivatives thereof, petroleum jelly, polydecenes or hydrogenated polyisobutene such as parleam; natural or synthetic essential oils such as, for example, eucalyptus oil, hybrid lavender oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil; fatty alcohols containing from 8 to 26 carbon atoms, such as cetyl alcohol, stearyl alcohol, and the mixture thereof (cetylstearyl alcohol), octyldodecanol, 2-butyloctanol, 2-hexyldecanol, 2-undecylpentadecanol, oleyl alcohol or linoleyl alcohol; partially hydrocarbon-based and/or silicone-based fluoro oils such as those described in document JP-A-2-295 912; silicone oils such as volatile or non-volatile polydimethylsiloxanes (PDMSs) containing a linear or cyclic silicone chain, which are liquid or pasty at room temperature, in particular cyclopolydimethylsiloxanes (cyclomethicones) such as cyclohexasiloxane; polydimethylsiloxanes comprising alkyl, alkoxy or phenyl groups, pendant or at the end of a silicone chain, these groups containing from 2 to 24 carbon atoms; phenylsilicones such as phenyl trimethicones, phenyl dimethicones, phenyltrimethyl-siloxydiphenylsiloxanes, diphenyl dimethicones, diphenylmethyldiphenyltrisiloxanes, 2-phenylethyl trimethylsiloxysilicates and polymethylphenylsiloxanes; mixtures thereof.

The term “hydrocarbon-based oil” in the list of abovementioned oils embraces any oil comprising predominantly carbon and hydrogen atoms, and optionally ester, ether, fluoro, carboxylic acid and/or alcohol groups.

When present, the amount of oil may range, for example, from about 0.01% to about 50% by weight, from about 0.1% to about 30% by weight, from about 0.1% to about 20% by weight, from about 0.1 to about 10% by weight, from about 0.1% to about 5% by weight based on the total weight of the composition.

Needless to say, the composition of the invention should be cosmetically or dermatologically acceptable, i.e., it should contain a non-toxic physiologically acceptable medium and should be able to be applied to the hair, skin, scalp, lips, nails, and eyelashes of human beings.

The compositions for application to keratinous substrates preferably constitute a composition for styling hair, conditioning hair, preventing hair frizz, making hair shinier or helping preserve the color of hair.

The compositions for application to keratinous substrates also preferably constitute a cosmetic or dermatological composition for protecting, treating, or caring for keratinous tissue, such as hair, including eyelashes and eyebrows, skin, nails and lips.

According to yet other preferred embodiments of the present disclosure, the composition of the present invention may be used as a carrier vehicle for various types of skin care active. Examples of suitable skin care benefits include photoprotection, moisturization, suppleness, softness, silkiness, desquamation, anti-aging, anti-wrinkle, reduction of fine lines, skin lightening, skin tightening, evening of skin tone, and anti-dandruff benefits to the scalp.

According to further embodiments of the present disclosure, methods of beneficially coating keratinous substrates comprising applying onto the keratinous substrates a composition comprising a silicone amine, an anionic silicone, and a cosmetically acceptable carrier, are provided.

According to other further embodiments of the present disclosure, methods of beneficially coating keratinous substrates comprising applying onto the keratinous substrates a composition comprising a silicone amine, an anionic silicone, a cosmetically acceptable carrier, and an auxiliary agent such as film forming agents, surfactants, conditioning agents, and skin active agents, are provided.

According to further preferred embodiments, methods of beneficially coating keratinous substrates with the compositions of the present disclosure are provided in order to impart benefits to keratinous substrates such as (1) shine to hair; (2) improving the condition/manageability of hair; (3) preventing hair from becoming frizzy, i.e., as an “anti-frizz” agent; (4) styling hair; (5) color retention or improving fade resistance of artificially colored hair, (6) moisture retention to skin, scalp or hair; (7) providing sun protection; and (8) providing skin care benefits such as, for example, anti-aging, whitening/depigmentation, anti-acne, etc.

The compositions and/or products containing the compositions of the present disclosure may be in the form of a cream, a lotion, a mousse or a gel.

The compositions and/or products containing the compositions of the present disclosure may also be in the form of an oil-in-water emulsion, a water-in-oil emulsion, an aqueous composition, a hydro-alcoholic composition, or an anhydrous composition.

The compositions and/or products containing the compositions of the present disclosure may also be anhydrous or may contain less than 3% of water.

Other embodiments include having the disclosed compositions in various hair styling and hair care products such as gels, mousses, sprays, hair lotions, hair masques, shampoos, conditioners, masques, and hair treatment products and applying such products onto hair. Such products may be rinse-off or leave-on products.

Additional embodiments include having the disclosed compositions in various skin care, skin treatment, scalp care and treatment, nail and lip care products such as moisturizers, skin peels, skin masques, anti-wrinkle products, anti-aging products, sunscreen products, body and facial washes and cleansers, and anti-dandruff shampoos. Such products may be rinse-off or leave-on products.

### EXAMPLES

### Example 1

### Shine Study

A test swatch of Regular Bleached hair from International Hair Importers “IHIP” (2 cm width, 19 cm length) was treated with 0.4 g of the silicone-based composition containing 30% Silicone phosphate, 40% Amodimethicone, and 30% water as a leave-in. Untreated hair was used as a control. Shine measurements were taken as Reich-Robbins values, using the SAMBA. SAMBA is an instrument used to analyze the visual appearance of hair. It uses a polarization camera and obtains image of the hair, which is then analyzed to determine the shine and chroma of the hair tress.

The final shine measurements for the Control and treated Test swatches were 18.031 and 100.837, respectively. The % change in shine was 459%. Therefore, the disclosed complex provided approximately 5 times more shine compared to the untreated hair.

### Example 2

### Durable Shine Study

A test swatch of IHIP Regular Bleached hair swatch (2 cm width, 19 cm length) was treated with 0.4 g of the silicone-based composition containing 30% Silicone phosphate, 40% Amodimethicone, and 30% water, then shampooed once using Sodium Lauryl Ether Sulfate-2 (SLES-2) 15% pH=6, 15 sec massage, 15 sec rinse (80 gph, 32° C.). Hair was dried completely. Untreated hair was used as a control. Shine measurements were taken as Reich-Robbins values, using the SAMBA. The final shine measurements for the Control and treated Test swatches were 18.031 and 37.843, respectively. The % change in shine was 109.9%. Therefore, the disclosed complex provided approximately 2 times more shine compared to the untreated hair which shows the durability of the disclosed complex through a shampooing process.

### Example 3

### Anionic Anti-Frizz Shampoo Study

Four IHIP Natural Level 6 hair swatches (0.3 g, 19 cm) were treated with the following shampoos.

Control

| | |
|---|---|
| SLES-2 | 12% |
| DI Water | Q.S. to 100% |

Test

| | |
|---|---|
| Silsense PE-100L ™ | 5% |
| Amodimethicone | 10% |
| SLES-2 | 12% |
| DI Water | Q.S. |

0.5 g of Control and Test shampoos were massaged into wet hair swatches for 15 seconds and allowed to stand for 1 minute. The hair swatches were rinsed for 10 second (80 gph, 32 C). The hair swatches were then combed to remove any knots and to align the hair fibers. After winding and securing the hair swatches onto pegboards to create a 2-D wave pattern, the swatches were placed into a 50° C. oven for 1 hour, then allowed to dry overnight in ambient conditions. The swatches were carefully removed from the pegboards and hung in a humidity chamber (90% RH). The area of frizz was observed initially and after 4 hours.

The results showed that the Control swatches exhibited more frizz than the Test swatches after 4 hours. Therefore, the hair shampooed with the disclosed complex exhibited less frizz compared to the hair shampooed with the control shampoo.

### Example 4

### Amphoteric Anti-Frizz Shampoo Study

Four IHIP Natural Level 6 hair swatches (0.3 g, 19 cm) were treated with the following shampoos.

Control

| | |
|---|---|
| Cocamidopropyl Hydroxysultaine | 12% |
| DI Water | Q.S. to 100% |

Test

| | |
|---|---|
| Silsense PE-100L ™ | 5% |
| Amodimethicone | 10% |
| Cocamidopropyl Hydroxysultaine | 12% |
| DI Water | Q.S. |

0.5 g of Control and Test shampoos were massaged into wet hair swatches for 15 seconds and allowed to stand for 1 minute. The hair swatches were rinsed for 10 second (80 gph, 32 C). The hair swatches were then combed to remove any knots and to align the hair fibers. After winding and securing the hair swatches onto pegboards to create a 2-D wave pattern, the swatches were placed into a 50° C. oven for 1 hour, then allowed to dry overnight in ambient conditions. The swatches were carefully removed from the pegboards and hung in a humidity chamber (90% RH). The area of frizz was observed initially and after 4 hours.

The results showed that the Control swatches exhibited more frizz than the Test swatches after 4 hours. Therefore, the hair shampooed with the disclosed complex exhibited less frizz compared to the hair shampooed with the control shampoo.

### Example 5

### Wet Comb Study

Three IHIP Regular Bleached hair swatches (1″ width, 5″ length) were treated with the following compositions.

Control

| | |
|---|---|
| Silsense PE-100L ™ | 20% |
| DI Water | Q.S. to 100% |

Test

| | |
|---|---|
| Silsense PE-100L ™ | 20% |
| Amodimethicone | 40% |
| DI Water | Q.S. |

*Note:
Amodimethicone 40% is non-miscible in water and therefore was not tested.

The initial wet combing of untreated control and test hair swatches was performed using the Instron, measuring break energy (g-in). 1 gram of Control composition and 1 gram of Test composition were thoroughly massaged into the control and test hair swatches and the hair swatches were allowed to stand for 5 minutes. The hair swatches were wetted and 0.4 grams of shampoo per gram of swatch (SLES-2 15%, pH=6) were massaged into the control and test hair swatches for 30 seconds. The hair swatches were then rinsed in water for 15 seconds (80 gph, 32° C.), The final Break Energy was measured and statistical analyses were performed on the measurements (t-test).

The results showed that for the hair treated with the Test composition, there was a statistically significant change from initial to final wet combing measurements, as shown by Break Energy values of 513.14 and 198.96, respectively. Lower break energy values correspond to less energy required to comb the hair. Therefore, the treatment of the hair with the disclosed complex significantly improved the wet combing properties of the hair as compared to those of the untreated hair as shown by the reduced force in combing of about 2.5 times.

In addition, the % change in Break Energy between the hair treated with the Test composition and hair treated with the Control composition was −58.1 and −10.48, respectively. The more negative the change, the smoother the hair. Therefore, the hair treated with the disclosed complex exhibited significantly better wet combing properties compared to the hair treated with the control composition, as shown by an improvement in wet combing of 5.5 times.

### Example 6

### Color Retention Study

Six IHIP Platinum Bleached hair swatches (1 cm width, 19 cm length) were dyed with Redken Hi Fusion R with Pro-Oxide 20 vol. developer at 1:1 ratio. The hair swatches were then shampooed 6 times with the following shampoos.

Test

| | |
|---|---|
| Silsense PE-100L ™ | 5% |
| DC2-8566 ™ | 10% |
| SLES-2 | 12% |
| DI Water | Q.S. 100% |

Control 1

| | |
|---|---|
| SLES-2 | 12% |
| DI Water | Q.S. 100% |

Control 2

| | |
|---|---|
| Silsense PE-100L ™ | 5% |
| SLES-2 | 12% |
| DI Water | Q.S. 100% |

Control 3

| | |
|---|---|
| DC2-8566 ™ | 10% |
| SLES-2 | 12% |
| DI Water | Q.S. 100% |

Initial LAB values of hair swatches using Konica Minolta Spectrophotometer were measured. 0.4 grams of Test shampoo and Control shampoo were massaged into Test and Control hair swatches for 15 seconds. The hair swatches were then rinsed in water for 15 seconds (80 gph, 32° C.). The shampoo and rinse cycle was repeated 6 times. The hair swatches were completely dried and the final LAB values were calculated. The ΔE and %ΔL values were calculated.

The %ΔL results are as shown below.

| Sample | % ΔL |
|---|---|
| Test | 7.29 |
| Control 1 | 45.10 |
| Control 2 | 40.42 |
| Control 3 | 36.66 |

The lower the %ΔL value, the darker the color of the hair. Using an ANOVA statistical test, the %ΔL value for Test shampoo was found to be significantly lower than %ΔL value for the Control shampoos 1, 2, and 3. Therefore, the disclosed composition, when present in the shampoo, helped retain the color on the hair after shampooing and washing.

### Example 7

### Oxidative Hair Dye Intensifier Study

Six swatches of regular bleached hair from International Hair Importers “IHIP” (5 mm width, 19 cm length) were dyed with the following formulas, each formula mixed 1:1 with a 20 volume developer composition (6% hydrogen peroxide), 20 g of mixture per swatch, for 40 minutes of processing time.

*Control Formula (traditional hair color)

| | |
|---|---|
| DI Water | Q.S. to 100% |
| Oxidative dyes: | 3.3% |
| Toluene 2,5 Diamine | |
| m-Hydroquinone | |
| m-aminophenol | |
| 2,4-Diaminophenyxyethanol HCl | |
| N,N-Bis(2-Hydroxyethyl)-p- | |
| Phenylenediamine Sulfate | |
| Surfactants, glycol, polymers, | 66.7% |
| emulsifying agents, alkalizing | |
| agents, neutralizers, reducing | |
| agent, pH adjusters, fragrance, | |
| vitamins, ceramide | |

Test formula A (with Ammonia)

| | |
|---|---|
| DI Water | Q.S. to 100% |
| Dimethicone PEG-8 phosphate** | 20.70% |
| Amodimethicone*** | 47.40% |
| NH4OH | 1.85% (active) |
| Oxidative dyes: | 3.3% |
| Toluene 2,5 Diamine | |
| m-Hydroquinone | |
| m-aminophenol | |
| 2,4-Diaminophenyxyethanol HCl | |
| N,N-Bis(2-Hydroxyethyl)-p- | |
| Phenylenediamine Sulfate | |

Test formula B (no Ammonia)

| | |
|---|---|
| DI Water | Q.S. to 100% |
| Dimethicone PEG-8 phosphate | 20.70% |
| Oxidative dyes: | 3.3% |
| Toluene 2,5 Diamine | |
| m-Hydroquinone | |
| m-aminophenol | |
| 2,4-Diaminophenyxyethanol HCl | |
| N,N-Bis(2-Hydroxyethyl)-p- | |
| Phenylenediamine Sulfate | |

*control formulas in this example and other examples in the present disclosure are traditional hair color formulas that do not contain silicone amine-anionic silicone complex
**Silsense ® PE-100
***DC2-8566 ™

After rinsing the hair for 1 minute, the swatches were shampooed once with sodium lauryl ether sulfate-2 (SLES-2), 15% by weight in water (pH=6, 1 g/g hair). Initial and final L*A*B* values were measured using the Konica Minolta Spectrophotometer; ΔE and % ΔL were calculated, and a statistical analysis test (ANOVA) was performed.

The ΔE and %ΔL results are as follows:

| Sample | ΔE | % ΔL |
|---|---|---|
| Control formula | 39.879 | −64.17 |
| Test formula A | 41.837 | −68.75 |
| Test formula B | 44.816 | −73.50 |

The higher the ΔE value, the greater the overall change in color of the hair. The lower the %ΔL value, the darker the color of the hair after dyeing. According to the ANOVA test, the overall color changes, ΔE, for the Test formula A and Test formula B were statistically significantly greater than the ΔE, for the Control formula. In addition, the %ΔL value for the Test formula A and Test formula B were statistically significantly lower than the %ΔL value for the control formula. Furthermore, Test formula B performed better than Test A with respect to both ΔE and %ΔL comparisons with the control formula.

Therefore, the results show that the hair swatches dyed with the formulas containing the silicone amine/anionic silicone combination were significantly darker in color and had significantly greater color intensity compared to the swatches dyed with the control formula. This indicates significantly better color deposit using the compositions of the present disclosure compared to the traditional hair color (control formula).

Furthermore, hair swatches dyed with Test formula B, which did not contain ammonia were significantly darker and had significantly greater color intensity compared to the swatches dyed with Test formula A, which contained.

### Example 8

### Oxidative Hair Dye Intensifier Study

### Red Binary Dye Color

A swatch each of unpermed and permed IHIP 90% Gray hair swatches (5 mm width, 13 cm length) were dyed with the following formulas below, mixed 1:1 with a 20 volume developer composition (6% hydrogen peroxide), 10 g of mixture per swatch, for 30 minutes of processing time. Note: Permed hair was permed with Matrix™ Opticare Normal to Resistant formula, 15 min process, 5 minute neutralization.

Control formula

| | |
|---|---|
| DI Water | Q.S. to 100% |
| Oxidative dyes: | 0.73% |
| 1-hydroxyethyl 4,5 diamino | |
| pyrazole sulfate | |
| 5-amino-o-cresol | |
| Surfactants, glycol, propylene | 74.37% |
| glycol alkyl ether, alkalizing | |
| agents, neutralizers, pH | |
| adjusters, fatty alcohols, | |
| isopropyl alcohol, fragrance, | |
| amino acids, preservative | |

Test formula

| | |
|---|---|
| DI Water | Q.S. to 100% |
| Dimethicone PEG-8 phosphate | 30% |
| Amodimethicone | 40% |
| Oxidative dyes: | 0.73% |
| 1-hydroxyethyl 4,5 diamino | |
| pyrazole sulfate | |
| 5-amino-o-cresol | |

After rinsing the hair for 1 minute, the swatches were shampooed once with sodium lauryl ether sulfate-2 (SLES-2), 15% by weight in water (pH=6, 1 g/g hair). The final L*A*B* values were measured using the Konica Minolta Spectrophotometer; ΔE values between control vs. test were calculated. The results showed that the 90% Gray Permed and Unpermed swatches had calculated ΔE values of 9.44 and 7.49, respectively.

A higher ΔE value indicates a greater color change, and a ΔE>3 is generally accepted as significant color difference. The ΔE measurements and visual observation of the swatches show that the swatches dyed with the formula containing the silicone amine/anionic silicone combination had significantly greater overall color changes in terms of having more intense and darker red color.

Therefore, the swatches treated with the formula containing the silicone amine/anionic silicone combination exhibited significantly better color deposit on 90% gray hair than the control formula.

### Example 9

### Oxidative Hair Dye Intensifier Study

### Red Binary Dye Color with Ammonia

A swatch each of unpermed and permed IHIP 90% Gray hair swatches (5 mm width, 13 cm length) were dyed with the following formulas below, mixed 1:1 with a 20 volume developer composition (6% hydrogen peroxide), 10 g of mixture per swatch, for 30 s minutes of processing time. Note: Permed hair was permed with Matrix™ Opticare Normal to Resistant formula, 15 min process, 5 minute neutralization.

Control formula

| | |
|---|---|
| DI Water | Q.S. to 100% |
| Oxidative dyes: | 0.73% |
| 1-hydroxyethyl 4,5 diamino | |
| pyrazole sulfate | |
| 5-amino-o-cresol | |
| Surfactants, glycol, polymers, | 67.54% |
| emulsifying agents, alkalizing | |
| agents, neutralizers, reducing | |
| agent, pH adjusters, fragrance, | |
| vitamins, ceramide | |

Test formula

| | |
|---|---|
| DI Water | Q.S. to 100% |
| Dimethicone PEG-8 phosphate | 20.0% |
| Amodimethicone | 50.0% |
| Ammonium Hydroxide | 2.0% |
| Oxidative dyes: | 0.73% |
| 1-hydroxyethyl 4,5 diamino | |
| pyrazole sulfate | |
| 5-amino-o-cresol | |

After rinsing the hair for 1 minute, the swatches were shampooed once with SLES-2, 15% by weight of water (pH=6, 1 g/g hair). The final L*A*B* values were measured using the Konica Minolta Spectrophotometer; ΔE between control vs. test were calculated. The results showed that the 90% Gray Permed and Unpermed swatches had calculated ΔE values of 3.48 and 3.84, respectively.

A higher ΔE value indicates a greater color change, and a ΔE>3 is accepted as significant color difference. The ΔE measurements and visual observation of the swatches show that the swatches dyed with the formula containing the silicone amine/anionic silicone combination had significantly greater overall color changes, in terms of having more intense and darker red color.

Therefore, the swatches, swatches treated with the formula containing the silicone amine/anionic silicone combination had significantly better color deposit on 90% gray hair than the control formula.

### Example 10

### Oxidative Hair Dye Intensifier Study

### Blue Binary Dye Color

A swatch each of unpermed and permed IHIP 90% Gray hair swatches (5 mm width, 13 cm length) were dyed with the following formulas below, mixed 1:1 with a 20 volume developer composition (6% hydrogen peroxide), 10 g of mixture per swatch, for 30 minutes of processing time. Note: Permed hair was permed with Matrix™ Opticare Normal to Resistant formula, 15 min process, 5 minute neutralization.

Control formula:

| | |
|---|---|
| DI Water | Q.S. to 100% |
| Oxidative dyes: | 0.72% |
| Toluene 2,5 Diamine | |
| 2,4-diamino phenoxyethanol HCl | |
| Surfactants, glycol, propylene | 74.38% |
| glycol alkyl ether, alkalizing | |
| agents, neutralizers, pH | |
| adjusters, fatty alcohols, | |
| isopropyl alcohol, fragrance, | |
| amino acids, preservative | |

Test formula:

| | |
|---|---|
| DI Water | Q.S. to 100% |
| Dimethicone PEG-8 phosphate | 30% |
| Amodimethicone | 40% |
| Oxidative dyes: | 0.72% |
| Toluene 2,5 Diamine | |
| 2,4-diamino phenoxyethanol HCl | |

After rinsing the hair for 1 minute, the swatches were shampooed once with SLES-15% (pH=6, 1 g/g hair). The final L*A*B* values were measured using the Konica Minolta Spectrophotometer; ΔE between control vs. test were calculated. The results showed that the 90% Gray Permed and Unpermed swatches had calculated ΔE values of 12.06 and 15.46, respectively.

A higher ΔE value indicates a greater color change, and a ΔE>3 is generally is accepted as significant color difference. The ΔE measurements and visual observation of the swatches show that the swatches dyed with the formula containing the silicone amine/anionic silicone combination had significantly greater overall color changes, by having more intense and darker blue color.

Therefore, the swatches, swatches treated with the formula containing the silicone amine/anionic silicone combination had significantly better color deposit on 90% gray hair than the control formula.

### Example 11

### Oxidative Hair Dye Intensifier Study

### Blue Binary Dye Color with Ammonia

A swatch each of unpermed and permed IHIP 90% Gray hair swatches (5 mm width, 13 cm length) were dyed with the following formulas below, mixed 1:1 with a 20 volume developer composition (6% hydrogen peroxide), 10 g of mixture per swatch, for 30 minute processing time. Note: Permed hair was permed with Matrix™ Opticare Normal to Resistant formula, 15 min process, 5 minute neutralization.

Control formula

| | |
|---|---|
| DI water | Q.S. to 100% |
| Oxidative dyes: | 0.72% |
| Toluene 2,5 Diamine | |
| 2,4-diamino phenoxyethanol HCl | |
| Surfactants, glycol, polymers, | 67.55% |
| emulsifying agents, alkalizing | |
| agents, neutralizers, reducing | |
| agent, pH adjusters, fragrance, | |
| vitamins, ceramide | |

Test formula

| | |
|---|---|
| DI Water | Q.S. to 100% |
| Dimethicone PEG-8 phosphate | 20.0% |
| Amodimethicone | 50.0% |
| Ammonium Hydroxide | 2.0% |
| Oxidative dyes: | 0.72% |
| Toluene 2,5 Diamine | |
| 2,4-diamino phenoxyethanol HCl | |

After rinsing the hair for 1 minute, the swatches were shampooed once with SLES-2, 15% by weight in water (pH=6, 1 g/g hair). The final L*A*B* values were measured using the Konica Minolta Spectrophotometer; ΔE between control vs. test were calculated. The results showed that the 90% Gray Permed and Unpermed swatches had calculated ΔE values of 3.65 and 6.14, respectively.

A higher ΔE value indicates a greater color change, and a ΔE>3 is accepted as significant color difference. The ΔE measurements and visual observation of the swatches show that the swatches dyed with the formula containing the silicone amine/anionic silicone combination had significantly greater overall color changes, by having more intense and darker blue color.

Therefore, the swatches treated with the formula containing the silicone amine/anionic silicone combination had significantly better color deposit on 90% gray hair than the control formula.

### Example 12

### Oxidative Hair Dye Intensifier Study

### Violet Binary Dye Color

A swatch each of unpermed and permed IHIP 90% Gray hair swatches (5 mm width, 13 cm length) were dyed with the following formulas below, mixed 1:1 with a 20 volume developer composition (6% hydrogen peroxide), 10 g of mixture per swatch, for 30 minutes of processing time. Note: Permed hair was permed with Matrix™ Opticare Normal to Resistant formula, 15 min process, 5 minute neutralization.

Control formula

| | |
|---|---|
| DI water | Q.S. to 100% |
| Oxiative dyes: | 1.16% |
| p-phenylene diamine | |
| 5-amino-o-cresol | |
| Surfactants, glycol, propylene | 73.94% |
| glycol alkyl ether, alkalizing | |
| agents, neutralizers, pH | |
| adjusters, fatty alcohols, | |
| isopropyl alcohol, fragrance, | |
| amino acids, preservative | |

Test

| | |
|---|---|
| DI Water | Q.S. to 100% |
| Dimethicone PEG-8 phosphate | 30% |
| Amodimethicone | 40% |
| Oxiative dyes: | 1.16% |
| p-phenylene diamine | |
| 5-amino-o-cresol | |

After rinsing the hair for 1 minute, the swatches were shampooed once with SLES-2, 15% weight in water (pH=6, 1 g/g hair). The final L*A*B* values were measured using the Konica Minolta Spectrophotometer; ΔE between control vs. test were calculated. The results showed that the 90% Gray Permed and Unpermed swatches had calculated ΔE values of 9.22 and 6.96, respectively.

A higher ΔE value indicates a greater color change, and a ΔE>3 is generally is accepted as significant color difference. The ΔE measurements and visual observation of the swatches show that the swatches dyed with the formula containing the silicone amine and anionic silicone had significantly greater overall color changes, by having more intense and darker purple color.

Therefore, the swatches treated with the formula containing the silicone amine/anionic silicone combination had significantly better color deposit on 90% gray hair than the control formula.

### Example 13

### Oxidative Hair Dye Intensifier Study

### Violet Binary Dye Color with Ammonia

A swatch each of unpermed and permed IHIP 90% Gray hair swatches (5 mm width, 13 cm length) were dyed with the following formulas below, mixed 1:1 with a 20 volume developer composition (6% hydrogen peroxide), 10 g of mixture per swatch, for 30 s minutes of processing time. Note: Permed hair was permed with Matrix™ Opticare Normal to Resistant formula, 15 min process, 5 minute neutralization.

Control

| | |
|---|---|
| DI water | Q.S. to 100% |
| Oxiative dyes: | 1.16% |
| p-phenylene diamine | |
| 5-amino-o-cresol | |
| Surfactants, glycol, polymers, | 67.11% |
| emulsifying agents, alkalizing | |
| agents, neutralizers, reducing | |
| agent, pH adjusters, fragrance, | |
| vitamins, ceramide | |

Test

| | |
|---|---|
| DI Water | Q.S. to 100% |
| Dimethicone PEG-8 phosphate | 20.0% |
| Amodimethicone | 50.0% |
| Ammonium Hydroxide | 2.0% |
| Oxiative dyes: | 1.16% |
| p-phenylene diamine | |
| 5-amino-o-cresol | |

After rinsing the hair for 1 minute, the swatches were shampooed once with SLES-2, 15% by weight in water (pH=6, 1 g/g hair). The final L*A*B* values were measured using the Konica Minolta Spectrophotometer; ΔE between control vs. test were calculated. The results showed that the 90% Gray Permed and Unpermed swatches had calculated ΔE values of 5.235 and 29.38, respectively.

A higher ΔE value indicates a greater color change, and a ΔE>3 is accepted as significant color difference. The ΔE measurements and visual observation of the swatches show that the swatches dyed with the formula containing the silicone amine/anionic silicone combination had significantly greater overall color changes, by having more intense and darker purple color.

Therefore, the swatches treated with the formula containing the silicone amine/anionic silicone combination had significantly better color deposit on 90% gray hair than the control formula.

### Example 14

### Oxidative Dye Concentration Study

A swatch each of IHIP Platinum Bleached hair swatches (1 cm width, 13 cm length) was dyed with the following formulas below for 30 minutes of processing time.

Control 1:

| | |
|---|---|
| DI water | Q.S. to 100% |
| Oxidative dyes: | 0.73% |
| 1-hydroxyethyl 4,5 diamino | |
| pyrazole sulfate | |
| 5-amino-o-cresol | |
| Surfactants, glycol, propylene | 74.37% |
| glycol alkyl ether, alkalizing | |
| agents, neutralizers, pH | |
| adjusters, fatty alcohols, | |
| isopropyl alcohol, fragrance, | |
| amino acids, ceramide, | |
| preservative | |

Control 2:

| | |
|---|---|
| DI water | Q.S. to 100% |
| Oxidative dyes: | 0.72% |
| Toluene 2,5 Diamine | |
| 2,4-diamino phenoxyethanol HCl | |
| Surfactants, glycol, propylene | 74.38% |
| glycol alkyl ether, alkalizing | |
| agents, neutralizers, pH | |
| adjusters, fatty alcohols, | |
| isopropyl alcohol, fragrance, | |
| amino acids, ceramide, | |
| preservative | |

Control 3:

| | |
|---|---|
| DI water | Q.S. to 100% |
| Oxidative dyes: | 1.16% |
| p-phenylene diamine | |
| 5-amino-o-cresol | |
| Surfactants, glycol, propylene | 73.94% |
| glycol alkyl ether, alkalizing | |
| agents, neutralizers, pH | |
| adjusters, fatty alcohols, | |
| isopropyl alcohol, fragrance, | |
| amino acids, preservative | |

Test 1:

| | |
|---|---|
| DI Water | Q.S. to 100% |
| Dimethicone PEG-8 phosphate | 30.0% |
| Amodimethicone | 40.0% |
| Oxidative dyes: | 0.365% |
| 1-hydroxyethyl 4,5 diamino | |
| pyrazole sulfate | |
| 5-amino-o-cresol | |

Test 2:

| | |
|---|---|
| DI Water | Q.S. to 100% |
| Dimethicone PEG-8 phosphate | 30.0% |
| Amodimethicone | 40.0% |
| Oxidative dyes: | 0.36% |
| Toluene 2,5 Diamine | |
| 2,4-diamino phenoxyethanol HCl | |

Test 3:

| | |
|---|---|
| DI Water | Q.S. to 100% |
| Dimethicone PEG-8 phosphate | 30.0% |
| Amodimethicone | 40.0% |
| Oxidative dyes: | 0.58% |
| p-phenylene diamine | |
| 5-amino-o-cresol | |

The hair swatches were rinsed for 1 min (80 gph, 32° C.) and dried completely. The final L*A*B* values were measured using the Konica Minolta Spectrophotometer, and ΔE values between control and test formulas were calculated.

The ΔE of Control 1 vs. Test 1 is 17.75, and visually, the swatches dyed with the test formula had a darker red color. The ΔE of Control 2 vs. Test 2 is 13.62, and visually, the swatches dyed with the test formula had a darker blue color. The ΔE of Control 3 vs. Test 3 is 4.61, and visually, the swatches dyed with the test formula had a darker violet color.

A high ΔE value indicates a greater color change, and a ΔE>3 is accepted as a significant color difference. The ΔE values and visual observations of the swatches show that the swatches dyed with the test formulas containing the silicone amine/anionic silicone combination had significantly more color than the swatches dyed with the control formulas,

Therefore, the results showed that the dye compositions containing the silicone amine/anionic silicone combination deposited more color onto hair compared to the control formulas despite the reduction of the dye concentration by 50% in the test formulas.

### Example 15

### Oxidative Dye Time Study

Traditional hair dyes are usually processed for 30 minutes (+/−10 minutes). Reducing the processing time drastically to 10 minutes (+/−5 minutes) would be more desirable, however the efficiency of the resulting color would be sacrificed, unless a high load of dye compounds are added to compensate for the color in conventional methods. By using the composition of the present disclosure, reducing the processing time did not adversely affect the efficiency of the resulting color, even when the levels of the dye compounds used were consistent with those in traditional hair dyes.

A swatch each of IHIP 90% Gray Permed, 90% Gray Unpermed, and Regular Bleached hair swatches was dyed with the following formulas below for 5, 10, and 15 minute processing times.

Control

| | |
|---|---|
| DI Water | Q.S. to 100% |
| Oxidative dyes: | 2.815% |
| Toluene 2,5 Diamine | |
| m-Hydroquinone | |
| m-aminophenol2,4- | |
| Diaminophenyxyethanol HCl | |
| N,N-Bis(2-Hydroxyethyl)-p- | |
| Phenylenediamine Sulfate | |
| Surfactants, glycol, polymers, | 67.17% |
| emulsifying agents, alkalizing | |
| agents, neutralizers, reducing | |
| agent, pH adjusters, fragrance, | |
| vitamins, ceramide | |

Test

| | |
|---|---|
| DI Water | Q.S. to 100% |
| Dimethicone PEG-8 phosphate | 20.70% |
| Amodimethicone | 47.40% |
| Oxidative dyes: | 2.815% |
| Toluene 2,5 Diamine | |
| m-Hydroquinone | |
| m-aminophenol2,4- | |
| Diaminophenyxyethanol HCl | |
| N,N-Bis(2-Hydroxyethyl)-p- | |
| Phenylenediamine Sulfate | |

The hair swatches were rinsed for 1 min (80 gph, 32° C.) and dried completely. The final L*A*B* values were measured using the Konica Minolta Spectrophotometer, and ΔE values between control and test formulas were calculated. Results are as follow:

5 minute processing time, 90% gray permed hair had ΔE of 14.95 and 90% gray unpermed hair had a ΔE value of 14.55.

10 minute processing time, 90% gray permed hair had ΔE of 11.06 and 90% gray unpermed hair had a ΔE value of 10.70.

15 minute processing time, 90% gray permed hair had ΔE of 9.63 and 90% gray unpermed hair had a ΔE value of 9.86.

A high ΔE value indicates a greater color change, and a ΔE>3 is accepted as significant color difference. The ΔE values and visual observations of the swatches show that the swatches dyed with the formulas containing the silicone amine/anionic silicone combination had significantly greater overall color change, by having more intense and darker color compared to the control formula.

Therefore, the formulas containing the silicone amine/anionic silicone combination provided better color deposit than the traditional dye at shorter processing times of 5, 10, or 15 minutes.

### Example 16

### Semi-Permanent Red Brown Dye Booster Study

Six IHIP Platinum Bleached hair swatches (2.5 g) were dyed with the following formulas below for 30 minutes of processing time.

Control Formula

| | | |
|---|---|---|
| Hair color containing | 5 | g |
| direct dyes and oxidative dyes: | | |
| basic red 51 | | |
| basic yellow 87 | | |
| HC blue no. 2 | | |
| HC red no. 3 | | |
| HC yellow no. 7 | | |
| 3-nitro-p-hydroxyethylaminophenol | | |
| 2-hydroxyethyamino-5-nitroanisole | | |
| Propylene glycol | | |
| amodimethicone | | |
| Surfactants, polymers, fragrance, | | |
| preservative, ceramide | | |

Test Formula

| | | |
|---|---|---|
| Hair color containing | 4 | g |
| direct dyes and oxidative dyes: | | |
| basic red 51 | | |
| basic yellow 87 | | |
| HC blue no. 2 | | |
| HC red no. 3 | | |
| HC yellow no. 7 | | |
| 3-nitro-p-hydroxyethylaminophenol | | |
| 2-hydroxyethyamino-5-nitroanisole | | |
| Propylene glycol | | |
| amodimethicone | | |
| Surfactants, polymers, fragrance, | | |
| preservative, ceramide | | |
| Dimethicone PEG-8 phosphate | 0.5 | g |
| Amodimethicone | 0.5 | g |

The hair swatches were rinsed for 1 min (80 gph, 32° C.) and dried completely. The initial and final L*A*B* values were measured using the Konica Minolta Spectrophotometer, and ΔE was calculated.

After treatment, the ΔE values for Test formula and Control formula were 50.222 and 41.278, respectively. The higher the ΔE value, the greater the overall change in color of the hair. In addition, %ΔL values for Test formula and Control formula were −57.845 and −70.513, respectively. The lower the %ΔL value, the darker the color of the hair. According to a statistical t-Test, the Test formula performed statistically significantly better than the Control formula in terms of both A E and %ΔL comparisons. Therefore, the addition of the silicone amine/anionic silicone combination to the dye composition helped increase the color deposit and boost the intensity of the color on the hair, despite the dilution of the dye concentration by 20% in the test formula.

### Example 17

### Semi-Permanent Red Dye Booster Study

A swatch each of IHIP Platinum Bleached hair swatches (2.5 g) was dyed with the following formulas below for 30 minutes of processing time.

Control Formula

| | | |
|---|---|---|
| Hair color containing | 5 | g |
| direct dyes and oxidative dyes: | | |
| basic red 51 | | |
| basic yellow 87 | | |
| HC orange no. 2 | | |
| 4-amino-3-nitrophenol | | |
| Propylene glycol | | |
| amodimethicone | | |
| Surfactants, polymers, fragrance, | | |
| preservative, ceramide | | |

Test Formula

| | | |
|---|---|---|
| Hair color containing | 4 | g |
| direct dyes and oxidative dyes: | | |
| basic red 51 | | |
| basic yellow 87 | | |
| HC orange no. 2 | | |
| 4-amino-3-nitrophenol | | |
| Propylene glycol | | |
| amodimethicone | | |
| Surfactants, polymers, fragrance, | | |
| preservative, ceramide | | |
| Dimethicone PEG-8 phosphate | 0.5 | g |
| Amodimethicone | 0.5 | g |

The hair swatches were rinsed for 1 min (80 gph, 32° C.) and dried completely. The final L*A*B* value was measured using the Konica Minolta Spectrophotometer, and ΔE was calculated between the Test formula and Control formula.

After treatment, the ΔE value for Test formula vs. Control formula was 18.03. A high ΔE value indicates a greater color change, and a ΔE>3 is accepted as significant color difference. The ΔE value and visual observation of the swatches show that the swatches dyed with the dye composition containing the silicone amine/anionic silicone combination had a significantly better overall color change, by having a more intense and darker red color. Therefore, the addition of the silicone amine/anionic silicone combination to the dye composition helped boost the intensity of the color on the hair, despite the dilution of the dye concentration by 20% in the test formula.

### Example 18

### Semi-Permanent Color Intensifier Study

IHIP Platinum Bleached hair swatches (1 cm width, 13 cm length) were dyed with the following formulas below, for 30 minutes of processing time (three swatches per formula).

Control Formula:

| | | |
|---|---|---|
| Surfactants, emulsifying agents, | 71 | g |
| polymers, alkalizing agent, | | |
| neutralizes glycol, water | | |
| Basic Red #51 | 0.5 | g |

| | |
|---|---|
| DI Water | Q.S. |

Test Formula:

| | | |
|---|---|---|
| Dimethicone PEG-8 phosphate | 20 | g |
| Amodimethicone | 40 | g |
| Basic Red #51 | 0.5 | g |

| | |
|---|---|
| DI Water | Q.S. |
| pH | 7.0 |

The hair swatches were rinsed for 2 minutes (100 gph, 32° C.) and dried completely. The final L*A*B* values were measured using the Konica Minolta Spectrophotometer; ΔE between control vs. test were calculated.

The results showed that hair dyed with the formula containing the silicone amine/anionic silicone combination had a more dark red color than the hair dyed with the control formula. The average ΔE value between the control and the test was 20.28, corresponding to a significantly higher overall change in color for the swatches dyed with the test formula. Therefore, the formula containing the silicone amine/anionic silicone combination provided a significantly more intense color to the hair compared to the traditional hair color base.

### Example 19

### Semi-Permanent Durable Color Study

Six IHIP Platinum Bleached hair swatches were dyed with the following formulas below, for 30 minutes of processing time.

Control Formula

| | | |
|---|---|---|
| Hair color containing | 5 | g |
| direct dyes and oxidative dyes: | | |
| basic red 51 | | |
| basic yellow 87 | | |
| HC blue no. 2 | | |
| HC red no. 3 | | |
| HC yellow no. 7 | | |
| 3-nitro-p- | | |
| hydroxyethylaminophenol | | |
| 2-hydroxyethyamino-5- | | |
| nitroanisole | | |
| Propylene glycol | | |
| amodimethicone | | |
| Surfactants, polymers, fragrance, | | |
| preservative, ceramide | | |

Test Formula

| | | |
|---|---|---|
| Hair color containing | 4 | g |
| direct dyes and oxidative dyes: | | |
| basic red 51 | | |
| basic yellow 87 | | |
| HC blue no. 2 | | |
| HC red no. 3 | | |
| HC yellow no. 7 | | |
| 3-nitro-p- | | |
| hydroxyethylaminophenol | | |
| 2-hydroxyethyamino-5- | | |
| nitroanisole | | |
| Propylene glycol | | |
| amodimethicone | | |
| Surfactants, polymers, fragrance, | | |
| preservative, ceramide | | |
| Dimethicone PEG-8 phosphate | 0.5 | g |
| Amodimethicone | 0.5 | g |

The hair swatches were rinsed for 1 minute (80 gph, 32° C.) and dried completely. Initial L*A*B* values were measured using the Konica Minolta Spectrophotometer. The swatches were then shampooed with 10% SLES-2 (15 seconds wash, 10 seconds rinsing). The shampooing and rinsing steps were repeated for a total of seven cycles of shampooing and rinsing. The swatches were dried completely and final L*A*B* values were measured. The ΔE and % change in L values (%ΔL) were calculated.

The results showed that hair dyed with the formula containing the silicone amine/anionic silicone combination had retained more color than the hair dyed with the control formula. Control and Test swatches had ΔE values of 20.52 and 27.98, respectively, after 7 shampoo/rinse cycles. The higher the ΔE, the greater the overall change in color of the hair. In addition, Control and Test swatches had %ΔL values of −22.89 and −33.4, respectively, after 7 shampoo cycles. The lower the change in L value, the darker the color of the hair. Therefore, the addition of the silicone amine/anionic silicone combination to the direct dye composition helped the dyed hair to substantially retain its color even after several shampoo/rinse cycles.

Having described the subject matter of the present disclosure by way of illustration and example for purposes of clarity of understanding, it will be apparent to one of ordinary skill in the art that the same can be performed by modifying or changing the subject matter within a variety of conditions, formulations and other parameters without affecting its scope or any specific embodiment thereof, and that such modifications or changes are intended to be encompassed within the scope of the appended claims. All publications, patents, and patent applications mentioned in this specification are indicative of the level of skill of those skilled in the art to which this invention pertains, and are herein incorporated by reference in their entirety to the same extent as if each individual publication, patent or patent application was specifically and individually indicated to be incorporated by reference.

1. A cosmetic composition comprising:
(a) at least one silicone amine;
(b) at least one anionic silicone; and
(c) a cosmetically acceptable carrier.

2. The cosmetic composition of claim 1, wherein the at least one silicone amine (a) is chosen from amodimethicone, trimethylsilylamodimethicone, aminopropyl phenyltrimethicone, PEG-7 amodimethicone, aminopropyl triethyoxysilane, alkyl modified amino silicone, amino silanes, PEG-8 amodimethicone, bis-isobutyl PEG-14/amodimethicone copolymer, bis-isobutyl PEG-15/amodimethicone copolymer, bis-isobutyl PEG/PPG-20/35/amodimethicone copolymer and mixtures thereof.

3. The cosmetic composition of claim 1, wherein the at least one silicone amine (a) includes amodimethicone.

4. The cosmetic composition of claim 1, wherein the at least one silicone amine (a) is present in an amount of from greater than 0 to about 90% by weight, based on the total weight of the cosmetic composition.

5. The cosmetic composition of claim 1, wherein the at least one silicone amine (a) is present in an amount of from about 1% to about 15% by weight, based on the total weight of the cosmetic composition.

6. The cosmetic composition of claim 1, wherein the at least one anionic silicone (b) is chosen from silicone carboxylates, silicone phosphates, silicone sulfates, silicone sulfosuccinates, silicone sulfonates, and mixtures thereof.

7. The cosmetic composition of claim 1, wherein the at least one anionic silicone (b) includes a silicone carboxylate.

8. The cosmetic composition of claim 1, wherein the silicone carboxylate is chosen from dimethicone PEG-7 phthalate and dimethicone PEG-7 succinate.

9. The cosmetic composition of claim 1, wherein the at least one anionic silicone (b) includes a silicone phosphate.

10. The cosmetic composition of claim 1, wherein the silicone phosphate is chosen from dimethicone PEG-7 phosphate, dimethicone PEG-8 phosphate, dimethicone PEG-10 phosphate, potassium dimethicone PEG-7 panthenyl phosphate, dimethicone PEG/PPG-7/4 phosphate, dimethicone PEG/PPG-12/4 phosphate, Trideceth-9 PG-Amodimethicone and mixtures thereof.

11. The cosmetic composition of claim 1, wherein the silicone phosphate includes dimethicone PEG-8 phosphate.

12. The cosmetic composition of claim 1, wherein the at least one anionic silicone (b) is present in an amount of from greater than 0 to about 90% by weight, based on the total weight of the cosmetic composition.

13. The cosmetic composition of claim 1, wherein the at least one anionic silicone (b) is present in an amount of from about 1% to about 15% by weight, based on the total weight of the composition.

14. The cosmetic composition of claim 1, wherein the cosmetically acceptable carrier (c) is chosen from water, alcohol, organic solvents, hydrocarbons, esters, silicones, and mixtures thereof.

15. The cosmetic composition of claim 1, wherein the cosmetically acceptable carrier (c) is present in an amount of from about 1% to about 90% by weight, based on the total weight of the cosmetic composition.

16. The cosmetic composition of claim 1, wherein the cosmetic composition further comprises at least one auxiliary ingredient (d) selected from the group consisting of film forming agents, surfactants, conditioning agents, skin active agents and mixtures thereof.

17. The cosmetic composition of claim 16 comprising at least one film forming agent, wherein the at least one film forming agent is selected from the group consisting of polysilicone 8, polydimethylsiloxane, dimethylsiloxane/3-thiopropyl methyl siloxane copolymer, vinylpyyrolidone/vinylacetate copolymer, polyvinyacetate, starch, polyquaternium-4, polyquaternium-11, acrylates/steareth-2 methacrylate crosspolymer, vinylacetate/vinyl neodecanoate copolymer, polyester-5, cetyl ethylhexanoate, vinyl acetate, crotonate/vinyl neodecanoate copolymer, 2-acryamido-2-methyl propane sulfonic acid (AMPS)/acrylic acid (AA) copolymer, AMPS/AA/acryl methacrylate copolymer, polyacrylamide, C₁₃-C₁₄ isoparaffin, laureth-7, octylacrylamide, acrylate/butylaminoethylmethacrylate copolymer and mixtures thereof.

18. The cosmetic composition of claim 16 comprising at least one surfactant, wherein the at least one surfactant is selected from the group consisting of anionic surfactants, cationic surfactants, nonionic surfactants, amphoteric surfactants, zwitterionic surfactants and mixtures thereof.

19. The cosmetic composition of claim 16 comprising at least one auxiliary ingredient, wherein the at least one auxiliary ingredient is a conditioning agent is selected from the group consisting of polyalkyl siloxane, a polyaryl siloxane, a polyalkylaryl siloxane, a polyether siloxane copolymer, amino acids, proteins, extracts, fats, oils, esters, hydrocarbons, quats, polyquats, polyols, humectants, alkanolamides, fatty acids, ketones and mixtures thereof.

20. The cosmetic composition of claim 16 comprising at least one skin active agent, wherein the at least one skin active agent is selected from the group consisting of photoprotective agents, self-tanning agents, desquamating agents, depigmenting agents, moisturizing agents, skin lightening agents, anti-aging ingredients, anti-wrinkle agents, anti-dandruff agents and mixtures thereof.

21. The cosmetic composition of claim 16, wherein the at least one auxiliary ingredient (d) is present in an amount of from about 0.05 to about 75% by weight, based on the total weight of the cosmetic composition.

22. A cosmetic composition comprising:
(a) at least one silicone amine chosen from amodimethicone, trimethylsilylamodimethicone, aminopropyl phenyltrimethicone, PEG-7 amodimethicone, aminopropyl triethyoxysilane, alkyl modified amino silicone, amino silanes, and mixtures thereof;
(b) at least one anionic silicone chosen from silicone carboxylates, silicone phosphates, silicone sulfates, silicone sulfosuccinates, silicone sulfonates, and mixtures thereof; and
(c) a cosmetically acceptable carrier.

23. The cosmetic composition of claim 22, further comprising at least one auxiliary ingredient (d) is chosen from film forming agents, surfactants, conditioning agents, skin active agents and mixtures thereof.

24. The cosmetic composition of claim 22, wherein
(a) the at least one silicone amine is amodimethicone;
(b) the at least one anionic silicone is chosen from silicone carboxylates, silicone phosphates, and mixtures thereof; and
(c) the cosmetically acceptable carrier comprises water.

25. The cosmetic composition of claim 22, wherein the at least one anionic silicone is dimethicone PEG-8 phosphate.

26. A method for beneficially coating a keratinous substrate comprising applying a composition according to claim 1 to the keratinous substrate.

27. A method for preserving artificial color on a keratinous substrate comprising applying a composition according to claim 1 to an artificially-colored keratinous substrate.

28. A hair coloring composition comprising:
(a) at least one silicone amine;
(b) at least one anionic silicone;
(c) at least one colorant;
(d) a cosmetically acceptable carrier; and
(e) optionally, at least one oxidizing agent.

29-59. (canceled)

60. A method for artificially coloring a keratinous substrate comprising applying a composition according to claim 28 to hair.

61. A method for simultaneously washing and depositing color onto hair comprising:
(a) applying a composition according to claim 29 to hair; and
(b) rinsing the composition from the hair.
